SEQUENCE LISTING (14 sequences)

SEQ ID NO: 1 - protein, 22 aa, Artificial sequence
  Signal Peptide for the ER
MKTNLFLFLIFSLLLSLSSAEF

SEQ ID NO: 2 - protein, 7 aa, Artificial sequence
  Vacuolar targeting signal from Tobacco chitinase A
DLLVDTM

SEQ ID NO: 3 - DNA, 21 bp, Artificial sequence
  Single strand DNA oligonucleotide
cagaattcgcccgcccctgca

SEQ ID NO: 4 - DNA, 22 bp, Artificial sequence
  Single strand DNA oligonucleotide
ctcagatcttggcgatgccaca

SEQ ID NO: 5 - DNA, 19 bp, Artificial sequence
  Single strand DNA oligonucleotide
ctcagaagaccagagggct

SEQ ID NO: 6 - DNA, 17 bp, Artificial sequence
  Single strand DNA oligonucleotide
caaagcggccatcgtgc

SEQ ID NO: 7 - DNA, 1491 bp, Homo sapiens
gcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcatcgccag

SEQ ID NO: 8 - protein, 497 aa, Homo sapiens [sequence omitted]

SEQ ID NO: 9 - DNA, 338 bp, Artificial sequence
  CaMV 35S Promoter nucleic acid sequence
ttttcacaaagggtaatatcgggaaacctcctcggattccattgcccagctatctgtcacttcatcgaaaggacagtagaaaaggaaggtggctcctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggac

SEQ ID NO: 10 - DNA, 66 bp, Artificial sequence
  Nucleic acid sequence encoding the ER signal peptide
atgaagactaatctttttctctttctcatcttttcacttctcctatcattatcctcggccgaattc

SEQ ID NO: 11 - DNA, 21 bp, Artificial sequence
  Nucleic acid sequence encoding the vacuolar targeting sequence
gatcttttagtcgatactatg

SEQ ID NO: 12 - DNA, 167 bp, Artificial sequence
  Sequence for terminator
taatttcatgatctgttttgttgtattcccttgcaatgcagggcctagggctatgaataaagttaatgtgtgaatgtgtgaatgtgtgattgtgacctgaagggatcacgactataatcgtttataataaacaaagactttgtcccaaaaaccccccccccngcaga

SEQ ID NO: 13 - DNA, 2186 bp, Artificial sequence
  Nucleic acid encoding recombinant GCD fused to signal peptides
ttttcacaaagggtaatatcgggaaacctcctcggattccattgcccagctatctgtcacttcatcgaaaggacagtagaaaaggaaggtggctcctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacaggcttcttgagatccttcaacaattaccaacaacaacaaacaacaaacaacattacaattactatttacaattacagtcgagggatccaaggagatataacaatgaagactaatctttttctctttctcatcttttcacttctcctatcattatcctcggccgaattcgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcatcgccaagatcttttagtcgatactatgtaatttcatgatctgttttgttgtattcccttgcaatgcagggcctagggctatgaataaagttaatgtgtgaatgtgtgaatgtgtgattgtgacctgaagggatcacgactataatcgtttataataaacaaagactttgtcccaaaaaccccccccccngcaga

SEQ ID NO: 14 - protein, 526 aa, Artificial sequence
  Recombinant GCD fused to signal peptides
MKTNLFLFLIFSLLLSLSSAEFARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQDLLVDTM

(19) United States
(12) Patent Application Publication
(10) Pub. No.: US 2005/0032211 A1
(43) Pub. Date: Feb. 10, 2005

(54) Cell/tissue culturing device, system and method

(75) Inventor: Yoseph Shaaltiel, Upper Galilee (IL)

Correspondence Address: G.E. EHRLICH (1995) LTD.;c/o ANTHONY CASTORINA, SUITE 207, 2001 JEFFERSON DAVIS HIGHWAY, ARLINGTON, VA, 22202, US

(73) Assignee: Metabogal Ltd.

(21) Appl. No.: 10/784,295

(22) Filed: Feb. 24, 2004

Related U.S. Application Data
(63) Continuation-in-part of application No. 10/121,534, filed on Apr. 12, 2002.
Application No. 10/121,534 is a continuation of application No. 09/246,600, filed on Feb. 8, 1999, now Pat. No. 6,391,638.
Application No. 09/246,600 is a continuation-in-part of application No. PCT/IL97/00316, filed on Sep. 26, 1997.

(30) Foreign Application Priority Data
Apr. 27, 2003 (IL) 155,588
Sep. 26, 1996 (IL) 119,310

(51) Int. Cl. C12N 5/00

(52) U.S. Cl. 435/420; 435/292.1; 435/296.1

(57) ABSTRACT

A device, system and method for axenically culturing and harvesting cells and/or tissues, including bioreactors and fermentors. The device is preferably disposable but nevertheless may be used continuously for a plurality of consecutive culturing/harvesting cycles prior to disposal of same. This invention also relates to batteries of such devices which may be used for large-scale production of cells and tissues. According to preferred embodiments of the present invention, the present invention is adapted for use with plant cell culture.

This Application is a Continuation-in-Part Application of, and claims priority from, U.S. patent application Ser. No. 10/121,534, filed on Apr. 12, 2002, which claims priority from U.S. patent application Ser. No. 09/246,600, filed on Feb. 8, 1999, now U.S. Pat. No. 6,391,638, which is a Continuation-in-Part Application of, and claims priority from, PCT Application No. PCT/IL97/00316, filed on Sep. 26, 1997, which claims priority from Israeli Patent Application No. 1193 10, filed on Sep. 26, 1996, all of which are hereby incorporated by reference as if fully set forth herein. This Application also claims priority from Israeli Patent Application No. 155,588, filed on Apr. 27, 2003, which is also hereby incorporated by reference as if fully set forth herein.

### FIELD OF THE INVENTION

The invention is of a device, system and method for cell/tissue culture, and in particular, of such a device, system and method for plant cell culture.

### BACKGROUND OF THE INVENTION

Cell and tissue culture techniques have been available for many years and are well known in the art. The prospect of using such culturing techniques economically is for the extraction of secondary metabolites, such as pharmaceutically active compounds, various substances to be used in cosmetics, hormones, enzymes, proteins, antigens, food additives and natural pesticides, from a harvest of the cultured cells or tissues. While potentially lucrative, this prospect has nevertheless not been effectively exploited with industrial scale bioreactors which use slow growing plant and animal cell cultures because of the high capital costs involved.

Background art technology for the production of cell and/or tissue culture at industrial scale, to be used for the production of such materials, is based on glass bioreactors and stainless steel bioreactors, which are expensive capital items. Furthermore, these types of industrial bioreactors comprise complicated and expensive mixing technologies such as impellers powered through expensive and complicated sterile seals; some expensive fermentors comprise an airlift multipart construction. Successful operation of these bioreactors often requires the implementation of aeration technologies which constantly need to be improved. In addition, such bioreactors are sized according to the peak volume capacity that is required at the time. Thus, problems arise when scaling up from pilot plant fermentors to large scale fermentors, or when the need arises to increase production beyond the capacity of existing bioreactors. The alternative to a large-capacity bioreactor, namely to provide a number of smaller glass or stainless steel bioreactors whose total volume capacity matches requirements, while offering a degree of flexibility for increasing or reducing overall capacity, is nevertheless much more expensive than the provision of a single larger bioreactor. Furthermore, running costs associated with most glass and stainless steel bioreactors are also high, due to low yields coupled to the need for sterilizing the bioreactors after every culturing cycle. Consequently, the products extracted from cells or tissues grown in such bioreactors are expensive, and cannot at present compete commercially with comparable products produced with alternative techniques. In fact, only one Japanese company is known to use the aforementioned cell/tissue culture technique commercially, using stainless steel bioreactors. This company produces Shikonin, a compound which is used almost exclusively in Japan.

Industrial scale, and even large scale, bioreactor devices are traditionally permanent or semi-permanent components, and no disclosure nor suggestion of the concept of a disposable bioreactor device for solving the aforementioned problems regarding large scale cell/tissue culture production is known of. On the contrary, disposable fermentors and bioreactor devices are well known and exclusively directed to very small scale production volumes, such as in home brewing and for laboratory work. These bioreactor devices generally comprise a disposable bag which is typically cut open in order to harvest the cell/tissue yield, thus destroying any further usefulness of the bag. One such known disposable bioreactor is produced by Osmotec, Israel, (Agritech Israel, issue No. 1, Fall 1997, page 19) for small-scale use such as in laboratory research. This bioreactor comprises a conical bag having an inlet through which culture medium, air, inoculant and other optional additives may be introduced, and has a volume of only about 1.5 liters. Aeration is performed by introducing very small air bubbles which in many cases results in damage to cells, particularly in the case of plant cell cultures. In particular, these bags are specifically designed for a single culture/harvest cycle only, and the bag contents are removed by cutting off the bottom of the bag. These bags are therefore not directed towards an economical solution to the question of providing industrial quantities of the materials to be extracted from the culture, as discussed above.

The term “disposable” in the present application means that the devices (bags, bioreactors etc.) are designed to be thrown away after use with only negligible loss. Thus devices made from stainless steel or glass are necessarily expensive devices and do not constitute a negligible loss for the operator of such devices. On the other hand, devices made from plastics such as flexible cheap plastics, for example, are relatively inexpensive and may therefore be, and are, disposed of after use with negligible economic loss. Thus, the disposability of these bioreactor devices does not generally present an economic disadvantage to the user, since even the low capital costs of these items is offset against ease of use, storage and other practical considerations. In fact, at the low production levels that these devices are directed, such is the economy of the devices that there is no motivation to increase the complexity of the device or its operation for the sake of enabling such a device to be used continuously for more than one culturing/harvesting cycle.

Further, sterile conditions outside the disposable bioreactor devices are neither needed nor possible in many cases, and thus once opened to extract the harvestable yield, it is neither cost-effective, practical nor often possible to maintain the opening sterile, leading to contamination of the bag and whatever contents may remain inside. Thus, these disposable devices have no further use after one culturing cycle.

Disposable bioreactor devices are thus relatively inexpensive for the quantities and production volumes which are typically required by non-industrial-scale users, and are relatively easy to use by non-professional personnel. In fact it is this aspect of simplicity of use and low economic cost, which is related to the low production volumes of the disposable devices, that is a major attraction of disposable bioreactor devices. Thus, the prior art disposable bioreactor devices have very little in common with industrial scale bioreactors—structurally, operationally or in the economics of scale—and in fact teach away from providing a solution to the problems associated with industrial scale bioreactors, rather than in any way disclosing or suggesting such a solution.

Another field in which some advances have been made in terms of experimental or laboratory work, while still not being useful for industrial-scale processes, is plant cell culture. Proteins for pharmaceutical use have been traditionally produced in mammalian or bacterial expression systems. In the past decade a new expression system has been developed in plants. This methodology utilizes Agrobacterium, a bacteria capable of inserting single stranded DNA molecules (T-DNA) into the plant genome. Due to the relative simplicity of introducing genes for mass production of proteins and peptides, this methodology is becoming increasingly popular as an alternative protein expression system (Ma, J. K. C., Drake, P. M. W., and Christou, P. (2003) Nature reviews 4, 794-805).

### SUMMARY OF THE INVENTION

The background art does not teach or suggest a device, system or method for industrial-scale production of materials through plant or animal cell culture with a disposable device. The background art also does not teach or suggest such a device, system or method for industrial-scale plant cell culture.

The present invention overcomes these deficiencies of the background art by providing a device, system and method for axenically culturing and harvesting cells and/or tissues, including bioreactors and fermentors. The device is preferably disposable but nevertheless may be used continuously for a plurality of consecutive culturing/harvesting cycles prior to disposal of same. This invention also relates to batteries of such devices which may be used for large-scale production of cells and tissues.

According to preferred embodiments of the present invention, the present invention is adapted for use with plant cell culture, for example by providing a low shear force while still maintaining the proper flow of gas and/or liquids, and/or while maintaining the proper mixing conditions within the container of the device of the present invention. For example, optionally and preferably the cells are grown in suspension, and aeration (flow of air through the medium, although optionally any other gas or gas combination could be used) is performed such that low shear force is present. To assist the maintenance of low shear force, optionally and preferably the container for containing the cell culture is made from a flexible material and is also at least rounded in shape, and is more preferably cylindrical and/or spherical in shape. These characteristics also optionally provide an optional but preferred aspect of the container, which is maintenance of even flow and even shear forces.

It should be noted that the term “plant culture” as used herein includes any type of transgenic and/or otherwise genetically engineered plant cell that is grown in culture. The genetic engineering may optionally be permanent or transient. Preferably, the culture features cells that are not assembled to form a complete plant, such that at least one biological structure of a plant is not present. Optionally and preferably, the culture may feature a plurality of different types of plant cells, but preferably the culture features a particular type of plant cell. It should be noted that optionally plant cultures featuring a particular type of plant cell may be originally derived from a plurality of different types of such plant cells.

The plant cell may optionally be any type of plant cell but is optionally and preferably a plant root cell (i.e. a cell derived from, obtained from, or originally based upon, a plant root), more preferably a plant root cell selected from the group consisting of Agrobacterium rihzogenes transformed root cell, celery cell, ginger cell, horseradish cell and carrot cell. Optionally and preferably, the plant cells are grown in suspension. The plant cell may optionally also be a plant leaf cell or a plant shoot cell, which are respectively cells derived from, obtained from, or originally based upon, a plant leaf or a plant shoot.

In a preferred embodiment, the plant root cell is a carrot cell. It should be noted that the transformed carrot cells of the invention are preferably grown in suspension. As mentioned above and described in the Examples, these cells were transformed with the Agrobacterium tumefaciens cells. According to a preferred embodiment of the present invention, any suitable type of bacterial cell may optionally be used for such a transformation, but preferably, an Agrobacterium tumefaciens cell is used for infecting the preferred plant host cells described below. Alternatively, such a transformation or transfection could optionally be based upon a virus, for example a viral vector and/or viral infection.

According to preferred embodiments of the present invention, there is provided a device for plant cell culture, comprising a disposable container for culturing plant cells. The disposable container is preferably capable of being used continuously for at least one further consecutive culturing/harvesting cycle, such that “disposable” does not restrict the container to only a single culturing/harvesting cycle. More preferably, the device further comprises a reusable harvester comprising a flow controller for enabling harvesting of at least a desired portion of the medium containing cells and/or tissues when desired, thereby enabling the device to be used continuously for at least one further consecutive culturing/harvesting cycle. Optionally and preferably, the flow controller maintains sterility of a remainder of the medium containing cells and/or tissue, such that the remainder of the medium remaining from a previous harvested cycle, serves as inoculant for a next culture and harvest cycle.

According to other embodiments of the present invention, there is provided a device, system and method which are suitable for culturing any type of cell and/or tissue. Preferably, the present invention is used for culturing a host cell. A host cell according to the present invention may optionally be transformed or transfected (permanently and/or transiently) with a recombinant nucleic acid molecule encoding a protein of interest or with an expression vector comprising the nucleic acid molecule. Such nucleic acid molecule comprises a first nucleic acid sequence encoding the protein of interest, optionally operably linked to one or more additional nucleic acid sequences encoding a signal peptide or peptides of interest. It should be noted that as used herein, the term “operably” linked does not necessarily refer to physical linkage.

“Cells”, “host cells” or “recombinant host cells” are terms used interchangeably herein. It is understood that such terms refer not only to the particular subject cells but also to the progeny or potential progeny of such a cell. Because certain modifications may occur in succeeding generation due to either mutation or environmental influences, such progeny may not, in fact, be identical to the parent cell, but are still included within the scope of the term as used herein. “Host cell” as used herein refers to cells which can be recombinantly transformed with naked DNA or expression vectors constructed using recombinant DNA techniques. As used herein, the term “transfection” means the introduction of a nucleic acid, e.g., naked DNA or an expression vector, into a recipient cells by nucleic acid-mediated gene transfer. “Transformation”, as used herein, refers to a process in which a cell's genotype is changed as a result of the cellular uptake of exogenous DNA or RNA, and, for example, the transformed cell expresses a recombinant form of the desired protein.

It should be appreciated that a drug resistance or other selectable marker is intended in part to facilitate the selection of the transformants. Additionally, the presence of a selectable marker, such as drug resistance marker may be of use in detecting the presence of contaminating microorganisms in the culture, and/or in the case of a resistance marker based upon resistance to a chemical or other factor, the selection condition(s) may also optionally and preferably prevent undesirable and/or contaminating microorganisms from multiplying in the culture medium. Such a pure culture of the transformed host cell would be obtained by culturing the cells under conditions which are required for the induced phenotype's survival.

As indicated above, the host cells of the invention may be transfected or transformed with a nucleic acid molecule. As used herein, the term “nucleic acid” refers to polynucleotides such as deoxyribonucleic acid (DNA), and, where appropriate, ribonucleic acid (RNA). The terms should also be understood to include. as equivalents. analogs of either RNA or DNA made from nucleotide analogs, and, as applicable to the embodiment being described, single-stranded (such as sense or antisense) and double-stranded polynucleotides.

In yet another embodiment, the host cell of the invention may be transfected or transformed with an expression vector comprising the recombinant nucleic acid molecule. “Expression Vectors”, as used herein, encompass vectors such as plasmids, viruses, bacteriophage, integratable DNA fragments, and other vehicles, which enable the integration of DNA fragments into the genome of the host. Expression vectors are typically self-replicating DNA or RNA constructs containing the desired gene or its fragments, and operably linked genetic control elements that are recognized in a suitable host cell and effect expression of the desired genes. These control elements are capable of effecting expression within a suitable host. Generally, the genetic control elements can include a prokaryotic promoter system or a eukaryotic promoter expression control system. Such system typically includes a transcriptional promoter, an optional operator to control the onset of transcription, transcription enhancers to elevate the level of RNA expression, a sequence that encodes a suitable ribosome binding site, RNA splice junctions, sequences that terminate transcription and translation and so forth. Expression vectors usually contain an origin of replication that allows the vector to replicate independently of the host cell.

Plasmids are the most commonly used form of vector but other forms of vectors which serves an equivalent function and which are or become known in the art are suitable for use herein. See, e.g., Pouwels et al. Cloning Vectors: a Laboratory Manual (1985 and supplements), Elsevier, N.Y.; and Rodriquez, et al. (eds.) Vectors: a Survey of Molecular Cloning Vectors and their Uses, Buttersworth, Boston, Mass. (1988), which are incorporated herein by reference.

In general, such vectors contain, in addition, specific genes which are capable of providing phenotypic selection in transformed cells. The use of prokaryotic and eukaryotic viral expression vectors to express the genes coding for the polypeptides of the present invention are also contemplated.

In one preferred embodiment, the host cell of the invention may be a eukaryotic or prokaryotic cell.

In a preferred embodiment, the host cell of the invention is a prokaryotic cell, preferably, a bacterial cell. In another embodiment, the host cell is a eukaryotic cell, such as a plant cell as previously described, or a mammalian cell.

The term “operably linked” is used herein for indicating that a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Optionally and preferably, operably linked DNA sequences are contiguous (e.g. physically linked) and, where necessary to join two protein-coding regions, in the same reading frame. Thus, a DNA sequence and a regulatory sequence(s) are connected in such a way as to permit gene expression when the appropriate molecules (e.g., transcriptional activator proteins) are bound to the regulatory sequence(s).

In another embodiment, this recombinant nucleic acid molecule may optionally further comprise an operably linked terminator which is preferably functional in the host cell, such as a terminator that is functional in plant cells. The recombinant nucleic acid molecule of the invention may optionally further comprise additional control, promoting and regulatory elements and/or selectable markers. It should be noted that these regulatory elements are operably linked to the recombinant molecule.

Regulatory elements that may be used in the expression constructs include promoters which may be either heterologous or homologous to the host cell, preferably a plant cell. The promoter may be a plant promoter or a non-plant promoter which is capable of driving high levels of transcription of a linked sequence in the host cell, such as in plant cells and plants. Non-limiting examples of plant promoters that may be used effectively in practicing the invention include cauliflower mosaic virus (CaMV) ³⁵S, rbcS, the promoter for the chlorophyll a/b binding protein, AdhI, NOS and HMG2, or modifications or derivatives thereof The promoter may be either constitutive or inducible. For example, and not by way of limitation, an inducible promoter can be a promoter that promotes expression or increased expression of the lysosomal enzyme nucleotide sequence after mechanical gene activation (MGA) of the plant, plant tissue or plant cell.

The expression vectors used for transfecting or transforming the host cells of the invention can be additionally modified according to methods known to those skilled in the art to enhance or optimize heterologous gene expression in plants and plant cells. Such modifications include but are not limited to mutating DNA regulatory elements to increase promoter strength or to alter the protein of interest.

The present invention therefore represents a revolutionary solution to the aforementioned problems of the background art, by providing a disposable bioreactor device for the large scale production of cell/tissue cultures. The device of the present invention, while essentially disposable, is characterized in comprising a reusable harvesting outlet for enabling harvesting of at least a portion of the medium containing cells and/or tissue when desired, thereby enabling the device to be used continuously for one or more subsequent consecutive culturing/harvesting cycles. In an industrial environment, sterility of the harvesting outlet during and after harvesting may be assured to a significantly high degree at relatively low cost, by providing, for example, a sterile hood in which all the necessary connections and disconnections of services to and from the device may be performed. When eventually the device does become contaminated it may then be disposed of with relatively little economic loss. Such devices may be cheaply manufactured, even for production volumes of 50 or 100 liters or more of culture. Further, the ability to perform a number of culturing/harvesting cycles is economically lucrative, lowering even further the effective cost per device.

A battery of such devices can be economically arranged, and the number of devices in the battery may be controlled to closely match production to demand. Thus, the transition from pilot plant bioreactors to large scale production may also be achieved in a relatively simple and economic manner by adding more devices to the battery. Further, the relatively low production volume of each device, coupled with the lack of solid mixers, results in relatively higher yields as compared to typical stainless steel bioreactors.

The device of the present invention therefore has a number of advantages over the background art, including but not limited to, being disposable; being economical to produce and simple to use; being disposable, but also being usable continuously for a plurality of consecutive cycles of culturing and harvesting desired cells and/or tissues; and optionally being suitable for operation according to a method in which inoculant is only required to be provided for the first culturing cycle, while inoculant for subsequent cycles is provided by a portion of the culture broth which remains in the device after harvesting same in a preceding cycle.

According to the present invention, there is provided a disposable device for axenically culturing and harvesting cells and/or tissue in at least one cycle, the device comprising a sterilisable disposable container having a top end and a bottom end, which container may be at least partially filled with a suitable sterile biological cell and/or tissue culture medium and/or axenic inoculant and/or sterile air and/or required other sterile additives, the container comprising: (i) a gas outlet for removing excess air and/or waste gases from the container; (ii) an additive inlet for introducing the inoculant and/or the culture medium and/or the additives into the container; and characterized in further comprising (iii) a reusable harvester comprising a flow controller for enabling harvesting of at least a desired portion of the medium containing cells and/or tissues when desired, thereby enabling the device to be used continuously for at least one further consecutive culturing/harvesting cycle, wherein a remainder of the medium containing cells and/or tissue, remaining from a previous harvested cycle, may serve as inoculant for a next culture and harvest cycle, wherein the culture medium and/or the required additives are provided.

Optionally, the disposable container is transparent and/or translucent. Also optionally the device further comprises an air inlet for introducing sterile gas in the form of bubbles into the culture medium through a first inlet opening, wherein the air inlet is connectable to a suitable gas supply. Preferably, the air inlet is for introducing sterile gas more than once during culturing. More preferably, the air inlet is for continuously introducing sterile gas. Optionally, a plurality of different gases are introduced at different times and/or concentrations through the air inlet.

Preferably, the harvester comprising a contamination preventer for substantially preventing introduction of contaminants into the container via the harvester.

Optionally, the container is non-rigid. Preferably, the container is made from a non-rigid plastic material. More preferably, the material is selected from the group comprising polyethylene, polycarbonate, a copolymer of polyethylene and nylon, PVC and EVA.

Optionally, the container is made from a laminate of more than one layer of the materials.

Also optionally, the container is formed by fusion bonding two suitable sheets of the material along predetermined seams.

Preferably, the air inlet comprises an air inlet pipe extending from the inlet opening to a location inside the container at or near the bottom end thereof

Also preferably, the at least one air inlet comprises a least one air inlet pipe connectable to a suitable air supply and in communication with a plurality of secondary inlet pipes, each the secondary inlet pipe extending to a location inside the container, via a suitable inlet opening therein, for introducing sterile air in the form of bubbles into the culture medium. More preferably, the device comprises a substantially box-like geometrical configuration, having an overall length, height and width. Most preferably, the height-to-length ratio is between about 1 and about 3, and preferably about 1.85. Optionally, the height to width ratio is between about 5 and about 30, and preferably about 13.

Preferably, the device comprises a support aperture substantially spanning the depth of the device, the aperture adapted to enable the device to be supported on a suitable pole support.

Optionally, the device further comprises a support structure for supporting the device. Preferably, the support structure comprises a pair of opposed frames, each the frame comprising upper and lower support members spaced by a plurality of substantially parallel vertical support members suitably joined to the upper and lower support members. More preferably, the plurality of vertical support members consists of at least one the vertical support member at each longitudinal extremity of the upper and lower support members.

Also more preferably, the frames are spaced from each other by a plurality of spacing bars releasably or integrally joined to the frames.

Also more preferably, the spacing bars are strategically located such that the device may be inserted and removed relatively easily from the support structure.

Optionally, the lower support member of each the frame comprises at least one lower support adapted for receiving and supporting a corresponding portion of the bottom end of the device.

Preferably, each the lower support is in the form of suitably shaped tab projecting from each of the lower support members in the direction of the opposed frame.

Optionally, the frames each comprise at least one interpartitioner projecting from each frame in the direction of the opposed frame, for to pushing against the sidewall of the device at a predetermined position, such that opposed pairs of the interpartitioner effectively reduce the width of the device at the predetermined position.

Preferably, the interpartitioner comprises suitable substantially vertical members spaced from the upper and lower support members in a direction towards the opposed frame with suitable upper and lower struts.

Optionally, the support structure may comprise a plurality of castors for transporting the devices.

Optionally, at least some of the air bubbles comprise a mean diameter of between about 1 mm and about 10 mm.

Also optionally, at least some of the air bubbles comprise a mean diameter of about 4 mm.

Optionally, the container comprises a suitable filter mounted on the gas outlet for substantially preventing introduction of contaminants into the container via the gas outlet.

Preferably, the container further comprises a suitable filter mounted on the additive inlet for substantially preventing introduction of contaminants into the container via the additive inlet.

Also preferably, there is a contamination preventer which comprises a U-shaped fluid trap, wherein one arm thereof is aseptically mounted to an external outlet of the harvester by suitable aseptic connector.

Preferably, the harvester is located at the bottom of the bottom end of the container.

Also preferably, the harvester is located near the bottom of the bottom end of the container, such that at the end of each harvesting cycle the remainder of the medium containing cells and/or tissue automatically remains at the bottom end of the container up to a level below the level of the harvester.

Optionally and preferably, the remainder of the medium containing cells and/or tissue is determined at least partially according to a distance d2 from the bottom of the container to the harvester.

Preferably, the remainder of the medium containing cells and/or tissue comprises from about 2.5% to about 45% of the original volume of the culture medium and the inoculant. More preferably, the remainder of the medium containing cells and/or tissue comprises from about 10% to about 20% of the original volume of the culture medium and the inoculant.

Optionally, the bottom end is substantially convex.

Also optionally, the bottom end is substantially frusta-conical.

Preferably, the container comprises an internal fillable volume of between about 5 liters and about 200 liters, preferably between about 50 liters and 150 liters, and preferably about 100 liters.

Optionally, the device further comprises suitable attacher for attaching the device to a suitable support structure. Preferably, the attacher comprises a loop of suitable material preferably integrally attached to the top end of the container.

According to preferred embodiments of the present invention, the device is adapted to plant cell culture. Preferably, the plant cell culture comprises plant cells obtained from a plant root. More preferably, the plant root is selected from the group consisting of Agrobacterium rihzogenes transformed root cell, celery cell, ginger cell, horseradish cell and carrot cell.

Optionally, there is provided a battery of the devices, comprising at least two the disposable devices as previously described. Preferably, the devices are supported by a suitable support structure via the attacher of each the device. Also preferably, the gas outlet of each the device is suitably connected to a common gas outlet piping which optionally comprises a blocker for preventing contaminants from flowing into the devices. Preferably, the blocker comprises a suitable filter.

Optionally, the additive inlet of each the device is suitably connected to a common additive inlet piping having a free end optionally comprising suitable aseptic connector thereat.

Optionally, the free end is connectable to a suitable supply of medium and/or additives.

Preferably, the harvester of each the device is suitably connected to a common harvesting piping having a free end optionally comprising suitable aseptic connector thereat.

More preferably, the battery further comprises a contamination preventer for substantially preventing introduction of contaminants into the container via the common harvesting piping. Preferably, the contamination preventer comprises a U-shaped fluid trap, wherein one arm thereof is free having an opening and wherein the other end thereof is aseptically mountable to the free end of the common harvesting piping via suitable aseptic connector.

More preferably, the free end of the U-tube is connectable to a suitable receiving tank.

Optionally, the air inlet of each the device is suitably connected to a common air inlet piping having a free end optionally comprising suitable aseptic connector thereat. Preferably, the free end is connectable to a suitable air supply.

According to other preferred embodiments of the present invention, there is provided a method for axenically culturing and harvesting cells and/or tissue in a disposable device comprising: providing the device which comprises a sterilisable transparent and/or translucent disposable container having a top end and a bottom end, which container may be at least partially filled with a suitable sterile biological cell and/or tissue culture medium and/or axenic inoculant and/or sterile air and/or other sterile required additives, the container comprising: (i) gas outlet for removing excess air and/or waste gases from the container; (ii) additive inlet for introducing the inoculant and/or the culture medium and/or the additives into the container; (iii) reusable harvester comprising suitable flow controller for enabling harvesting of at least a portion of the medium containing cells and/or tissue when desired, thereby enabling the device to be used continuously for at least one further consecutive cycle, wherein a remainder of the medium containing cells and/or tissue, remaining from a previously harvested cycle may serve as inoculant for a next culture and harvest cycle, wherein the culture medium and/or the required additives are provided; providing axenic inoculant via the harvester; providing sterile the culture medium and/or, sterile the additives via the additive inlet; optionally illuminating the container with external light; and allowing the cells and/or tissue to grow in the medium to a desired yield.

Preferably, the method further comprises: allowing excess air and/or waste gases to leave the container continuously via the gas outlet.

More preferably, the method further comprises: checking for contaminants and/or the quality of the cells/tissues which are produced in the container: if contaminants are found or the cells/tissues which are produced are of poor quality, the device and its contents are disposed of; if contaminants are not found, harvesting the desired portion of the medium containing cells and/or tissue.

Most preferably, while harvesting the desired portion, leaving a remainder of medium containing cells and/or tissue in the container, wherein the remainder of medium serves as inoculant for a next culture/harvest cycle. Also most preferably, the method further comprises: providing sterile the culture medium and/or sterile the additives for the next culture/harvest cycle via the additive inlet; and repeating the growth cycle until the contaminants are found or the cells/tissues which are produced are of poor quality, whereupon the device and its contents are disposed of

Preferably, the device further comprises an air inlet for introducing sterile air in the form of bubbles into the culture medium through a first inlet opening connectable to a suitable sterile air supply, the method further comprising the step of providing sterile air to the air inlet during the first and each subsequent cycle. More preferably, the sterile air is supplied continuously throughout at least one culturing cycle.

Also more preferably, the sterile air is supplied in pulses during at least one culturing cycle.

According to still other preferred embodiments of the present invention, there is provided a method for axenically culturing and harvesting cells and/or tissue in a battery of disposable devices comprising: providing a battery of devices as described above, and for at least one the device thereof: providing axenic inoculant to the device via the common harvesting piping; providing sterile the culture medium and/or sterile the additives to the device via the common additive inlet piping; optionally illuminating the device with external light; and allowing the cells and/or tissue in the device to grow in the medium to a desired yield.

Preferably, the method further comprises: allowing excess air and/or waste gases to leave the device continuously via the common gas outlet piping; checking for contaminants and/or the quality of the cells/tissues which are produced in the device: if in the device contaminants are found or the cells/tissues which are produced are of poor quality, the harvester of the device is closed off preventing contamination of other the devices of the battery; if in all of the devices of the battery contaminants are found or the cells/tissues which are produced therein are of poor quality, all the devices and their contents are disposed of; if contaminants are not found and the quality of the produced cells/tissues is acceptable, for each harvestable device, harvesting a desired portion of the medium containing cells and/or tissue via the common harvesting piping and the contamination preventer to a suitable receiving tank.

Preferably, a remainder of medium containing cells and/or tissue remains in the container, wherein the remainder serves as inoculant for a next culture/harvest cycle; and the method further comprises: providing sterile the culture medium and/or sterile the additives for the next culture/harvest cycle via the additive inlet.

Also preferably, the growth cycle is repeated until the contaminants are found or the cells/tissues which are produced are of poor quality for all of the devices of the battery, whereupon the contamination preventer is disconnected from the common harvester and the devices and their contents are disposed of

According to yet other preferred embodiments of the present invention, there is provided a method for axenically culturing and harvesting cells and/or tissue in a battery of disposable devices comprising: providing a battery of devices as described above, and for at least one the device thereof: providing axenic inoculant to the device via the common harvesting piping; providing sterile the culture medium and/or sterile the additives to the device via the common additive inlet piping; providing sterile air to the device via the common air inlet piping; optionally illuminating the device with external light; and allowing the cells and/or tissue in the device to grow in the medium to a desired yield.

Preferably, the method further comprises: allowing excess air and/or waste gases to leave the device continuously via the common gas outlet piping; and checking for contaminants and/or the quality of the cells/tissues which are produced in the device: if in the device contaminants are found or the cells/tissues which are produced are of poor quality, the harvester of the device is closed off preventing contamination of other the devices of the battery; if in all of the devices of the battery contaminants are found or the cells/tissues which are produced therein are of poor quality, all the devices and their contents are disposed of; if contaminants are not found and the quality of the produced cells/tissues is acceptable, the device is considered harvestable.

More preferably, the method further comprises: harvesting at least a desired portion of the medium containing cells and/or tissue for each harvestable device via the common harvesting piping and the contamination preventer to a suitable receiving tank.

Most preferably, a remainder of medium containing cells and/or tissue remains in the container, wherein the remainder serves as inoculant for a next culture/harvest cycle; and the method further comprises: providing sterile the culture medium and/or sterile the additives for the next culture/harvest cycle via the additive inlet.

Also most preferably, the growth cycle is repeated until the contaminants are found or the cells/tissues which are produced are of poor quality for all of the devices of the battery, whereupon the contamination preventer is disconnected from the common harvester and the devices and their contents are disposed of.

According to still other embodiments of the present invention, there is provided a device for plant cell culture, comprising a disposable container for culturing plant cells. Preferably, the disposable container is capable of being used continuously for at least one further consecutive culturing/harvesting cycle. More preferably, the device further comprises: a reusable harvester comprising a flow controller for enabling harvesting of at least a desired portion of the medium containing cells and/or tissues when desired, thereby enabling the device to be used continuously for at least one further consecutive culturing/harvesting cycle. Most preferably, the flow controller maintains sterility of a remainder of the medium containing cells and/or tissue, such that the remainder of the medium remaining from a previous harvested cycle, serves as inoculant for a next culture and harvest cycle.

According to yet other embodiments of the present invention, there is provided a method for culturing plant cells, comprising: culturing plant cells in a disposable container.

Preferably, the disposable container comprises an air inlet for introducing sterile gas or a combination of gases.

More preferably, the sterile gas comprises air. Most preferably, the sterile gas combination comprises a combination of air and additional oxygen.

Preferably, the oxygen is added separately from the air.

More preferably, the oxygen is added a plurality of days after initiating cell culture.

Preferably, the sterile gas or combination of gases is added more than once during culturing.

Also preferably, the air inlet is for continuously introducing sterile gas.

Also preferably, a plurality of different gases are introduced at different times and/or concentrations through the air inlet.

Preferably, the method further comprises: aerating the cells through the inlet. More preferably, the aerating comprises administering at least 1.5 L gas per minute.

Optionally and preferably, the method further comprises: providing sufficient medium for growing the cells. More preferably, sufficient medium is at a concentration of at least about 125% of a normal concentration of medium.

Preferably, the method further comprises: adding media during growth of the cells but before harvesting. More preferably, the method further comprises adding additional media at least about 3 days after starting culturing the cells.

Preferably, the method further comprises: replacing media completely at least about 3 days after starting culturing the cells.

Also preferably, the medium comprises a mixture of sugars.

Also preferably, the medium comprises a larger amount of sucrose than normal for cell culture.

Preferably, the plant cells produce a recombinant protein.

### BRIEF DESCRIPTION OF THE DRAWINGS

The invention is herein described, by way of example only, with reference to the accompanying drawings, wherein:

FIGS. 1A-C illustrate the main components of a first embodiment of the device of the present invention in front elevation and in cross-sectional side view, respectively for FIGS. 1A and 1B, and an exemplary system according to the present invention for FIG. 1C;

FIGS. 2a and 2b illustrate the main components of a second embodiment of the device of the present invention in front elevation and in cross-sectional side view, respectively;

FIG. 3 illustrates the main components of a third embodiment of the device of the present invention in cross-sectional side view;

FIG. 4 illustrates the seam lines of the first embodiment of the device of the present invention in front elevation;

FIGS. 5a and 5b illustrate the main components of a fourth embodiment of the device of the present invention in side view and in cross-sectional top view, respectively;

FIGS. 5(c) and 5(d) illustrate transverse cross-sections of the fourth embodiment taken along lines B-B and C-C in FIG. 5(a);

FIGS. 6a and 6b illustrate the main components of a fifth embodiment of the device of the present invention in side view and in cross-sectional top view, respectively;

FIGS. 6(c) and 6(d) illustrate transverse cross-sections of the fifth embodiment taken along lines B-B and C-C in FIG. 6(a);

FIG. 7 illustrates the embodiment of FIG. 5 in perspective view;

FIG. 8 illustrates the embodiment of FIG. 6 in perspective view;

FIG. 9 illustrates a support structure for use with the embodiments of FIGS. 5 to 8;

FIG. 10 illustrates the main components of a preferred embodiment of the battery of the present invention comprising a plurality of devices of any one of FIGS. 1 to 8;

FIGS. 11A and 11B show an expression cassette and vector for use with the present invention;

FIG. 12 shows growth of transformed (Glucocerebrosidase (GCD)) carrot cell suspension in a device according to the present invention as opposed to an Erlenmeyer flask;

FIG. 13 shows the relative amount of GCD produced by the device according to the present invention as opposed to an Erlenmeyer flask;

FIG. 14 shows the start point of 7% and 15% packed cell volume with regard to the growth curves, which are parallel;

FIG. 15 shows the amount of GCD protein from a quantitative Western blot for these two growth conditions;

FIG. 16 shows growth over an extended period of time (14 days) to find the stationary point;

FIG. 17 shows that the maximum amount of GCD (relative to other proteins) is produced by transformed cells through day 8, after which the amount of GCD produced starts to decline;

FIG. 18 shows that the replacement of media and/or the addition of fresh media on the fourth day maintains high growth level of cells beyond day 8, while FIGS. 19 and 20 show the amount of GCD produced under these conditions;

FIG. 21 shows the effect of different sugar regimes on cell growth;

FIG. 22 shows the effect of different sugar regimes on production of GCD;

FIGS. 23A and 23B show the effect of aeration rate on cell growth in a 10 L device according to the present invention; and

FIG. 24 shows the effect of adding more oxygen to the device according to the present invention.

### DETAILED DESCRIPTION OF THE PREFERRED EMBODIMENTS

The present invention is of a device, system and method for axenically culturing and harvesting cells and/or tissues, including bioreactors and fermentors. The device is preferably disposable but nevertheless may be used continuously for a plurality of consecutive culturing/harvesting cycles prior to disposal of same. This invention also relates to batteries of such devices which may be used for large-scale production of cells and tissues.

According to preferred embodiments of the present invention, the present invention is adapted for use with plant cell culture, as described above.

It should be noted that the term “plant culture” as used herein includes any type of transgenic and/or otherwise genetically engineered plant cell that is grown in culture. The genetic engineering may optionally be permanent or transient. Preferably, the culture features cells that are not assembled to form a complete plant, such that at least one biological structure of a plant is not present. Optionally and preferably, the culture may feature a plurality of different types of plant cells, but preferably the culture features a particular type of plant cell. It should be noted that optionally plant cultures featuring a particular type of plant cell may be originally derived from a plurality of different types of such plant cells.

The plant cell may optionally be any type of plant cell but is preferably a plant root cell selected from the group consisting of Agrobacterium rihzogenes transformed root cell, celery cell, ginger cell, horseradish cell and carrot cell. Optionally and preferably, the plant cells are grown in suspension.

In a preferred embodiment, the plant root cell is a carrot cell. It should be noted that the transformed carrot cells of the invention are preferably grown in suspension. As mentioned above and described in the Examples, these cells were transformed with the Agrobacterium tumefaciens cells. According to a preferred embodiment of the present invention, any suitable type of bacterial cell may optionally be used for such a transformation, but preferably, an Agrobacterium tumefaciens cell is used for infecting the preferred plant host cells described below.

According to preferred embodiments of the present invention, there is provided a device for plant cell culture, comprising a disposable container for culturing plant cells. The disposable container is preferably capable of being used continuously for at least one further consecutive culturing/harvesting cycle, such that “disposable” does not restrict the container to only a single culturing/harvesting cycle. More preferably, the device further comprises a reusable harvester comprising a flow controller for enabling harvesting of at least a desired portion of the medium containing cells and/or tissues when desired, thereby enabling the device to be used continuously for at least one further consecutive culturing/harvesting cycle. Optionally and preferably, the flow controller maintains sterility of a remainder of the medium containing cells and/or tissue, such that the remainder of the medium remaining from a previous harvested cycle, serves as inoculant for a next culture and harvest cycle.

According to optional embodiments of the present invention, the device, system and method of the present invention are adapted for mammalian cell culture, preferably for culturing mammalian cells in suspension. One of ordinary skill in the art could easily adapt the protocols and device descriptions provided herein for mammalian cell culture.

In one preferred embodiment, the host cell of the invention may be a eukaryotic or prokaryotic cell.

In a preferred embodiment, the host cell of the invention is a prokaryotic cell, preferably, a bacterial cell. In another embodiment, the host cell is a eukaryotic cell, such as a plant cell as previously described, or a mammalian cell.

Disclosed and described, it is to be understood that this invention is not limited to the particular examples, process steps, and materials disclosed herein as such process steps and materials may vary somewhat. It is also to be understood that the terminology used herein is used for the purpose of describing particular embodiments only and not intended to be limiting since the scope of the present invention will be limited only by the appended claims and equivalents thereof.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

It must be noted that, as used in this specification and the appended claims, the singular forms “a”, “an” and “the” include plural referents unless the content clearly dictates otherwise.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLE 1

### Illustrative Device

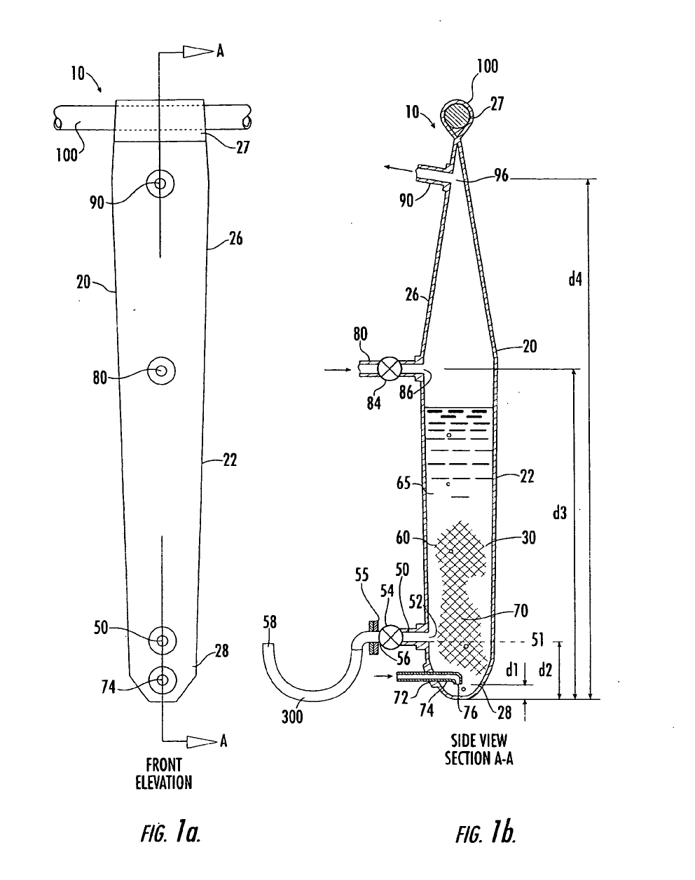

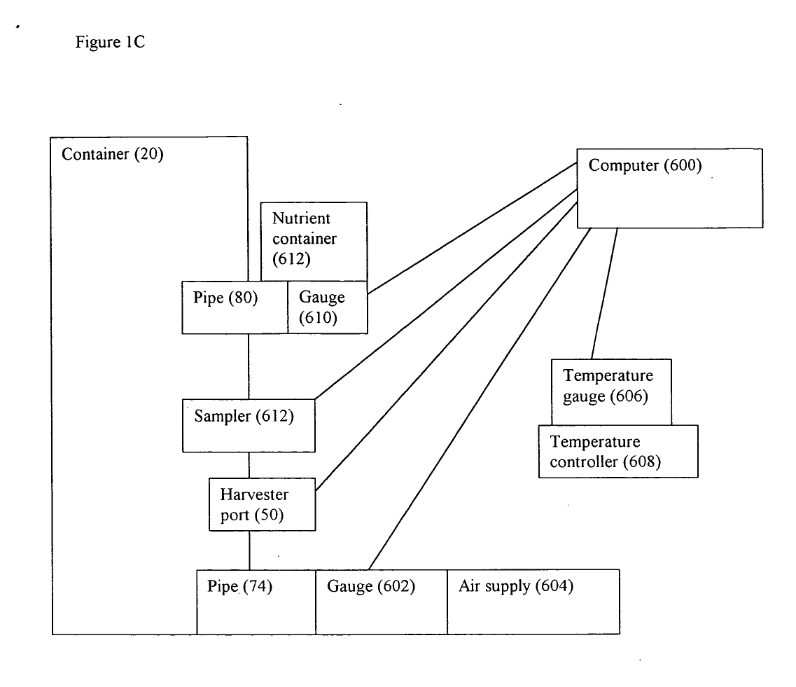

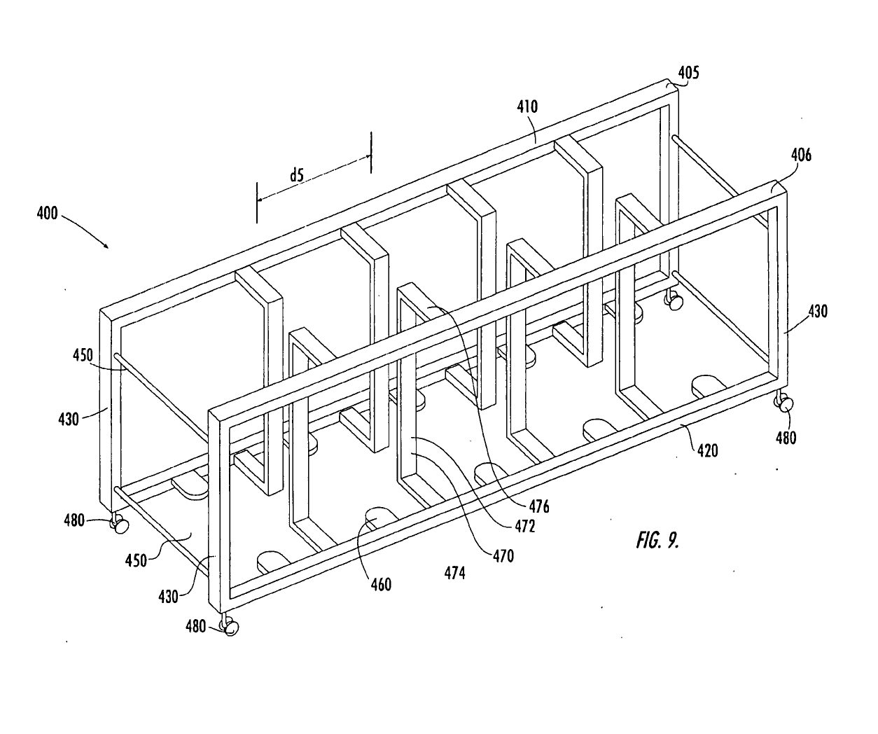

The principles and operation of the present invention may be better understood with reference to the drawings and the accompanying description. FIGS. 1-9 show schematic illustrations of various exemplary embodiments of the device according to the present invention.

It should be noted that the device according to the present invention, as described in greater detail below, may optionally feature all components during manufacture and/or before use. Alternatively, such components may be generated at the moment of use by conveniently combining these components. For example, any one or more components may optionally be added to the device to generate the complete device at the moment of use.

Referring now to the drawings, FIGS. 1, 2, and 3, correspond respectively to a first, second and third embodiments of the device, the device, generally designated (10), comprises a transparent and/or translucent container (20), having a top end (26) and a bottom end (28). The container (20) comprises a side wall (22) which is preferably substantially cylindrical, or at least features a rounded shape, though other shapes such as rectangular or polyhedral, for example, may also be suitable. Preferably, the bottom end (28) is suitably shaped to minimize sedimentation thereat. For example, in the first embodiment, the bottom end (28) is substantially frustro-conical or at least comprises upwardly sloping walls. In the second embodiment, the bottom end (28) comprises one upwardly sloping wall (29). In the third embodiment, the bottom end (28) is substantially cylindrical or alternatively convex. The aforementioned configurations of the bottom end (28), in conjunction with the location of the outlet (76) (hereinafter described) near the bottom end (28), enables air supplied via outlet (76) to induce a mixing motion to the container contents at the bottom end (28) which effectively minimizes sedimentation thereat. Nevertheless, the bottom end may be substantially flat in other embodiments of the present invention. The container (20) comprises an internal fillable volume (30) which is typically between 5 and 50 liters, though device (10) may alternatively have an internal volume greater than 50 liters or less than 5 liters. internal volume (30) may be filled with a suitable sterile biological cell and/or tissue culture medium (65) and/or axenic inoculant (60) and/or sterile air and/or required other sterile additives such as antibiotics or fungicides for example, as hereinafter described. In the aforementioned embodiments, the container (20) is substantially non-rigid, being made preferably from a non-rigid plastics material chosen from the group comprising polyethylene, polycarbonate, a copolymer of polyethylene and nylon, PVC and EVA, for example. Optionally, the container (20) may be made from a laminate of more than one layer of materials.

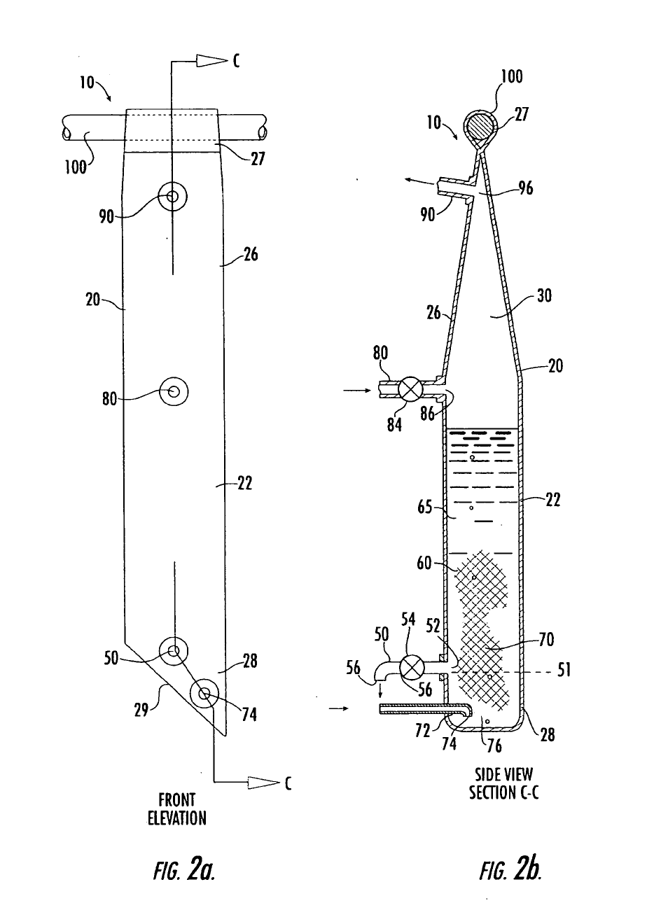

As shown for the third embodiment in FIG. 3, the container (20) may optionally comprise two concentric outer walls (24) to enhance mechanical strength and to minimize risk of contamination of the contents via the container walls.

In the first, second and third embodiments, device (10) is for aerobic use. Thus the container (20) further comprises at least one air inlet for introducing sterile air in the form of bubbles (70) into culture medium (65) through at least one air inlet opening (72). In the aforementioned embodiments, air inlet comprises at least one pipe (74) connectable to a suitable air supply (not shown) and extending from inlet opening (72) to a location inside container (20) at a distance d1 from the bottom of bottom end (28), wherein d1 may be typically around 1 cm, though it could be greater or smaller than 1 cm. The pipe (74) may be made from silicon or other suitable plastic material and is preferably flexible. The pipe (74) thus comprises an air outlet (76) of suitable diameter to produce air bubbles (70) of a required mean diameter. These bubbles not only aerate the medium (65), but also serve to mix the contents of the container, thereby minimizing sedimentation at the bottom end (28) as well, as hereinbefore described. The size of the bubbles delivered by the air inlet will vary according to the use of the device, ranging from well under 1 mm to over 10 mm in diameter. In some cases, particularly relating to plant cells, small bubbles may actually damage the cell walls, and a mean bubble diameter of not less than 4 mm substantially overcomes this potential problem. In other cases, much smaller bubbles are beneficial, and a sparger may be used at the air outlet (76) to reduce the size of the bubbles. In yet other cases air bubbles of diameter 10 mm or even greater may be optimal. Optionally, outlet (76) may be restrained in position at bottom end (28) through of a tether (not shown) or other means known in the art.

In other embodiments, device (10) is for anaerobic use, and thus does not comprise the air inlet.

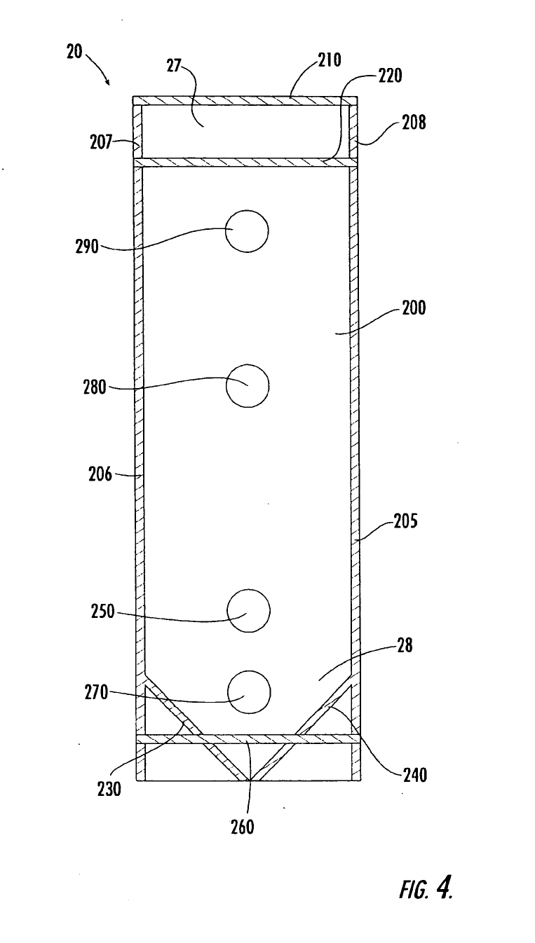

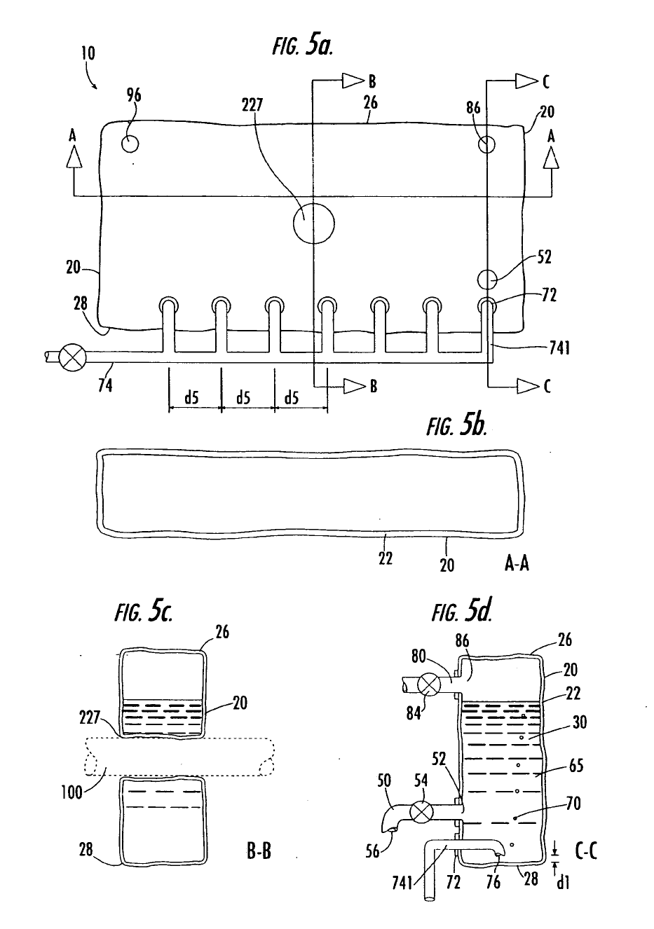

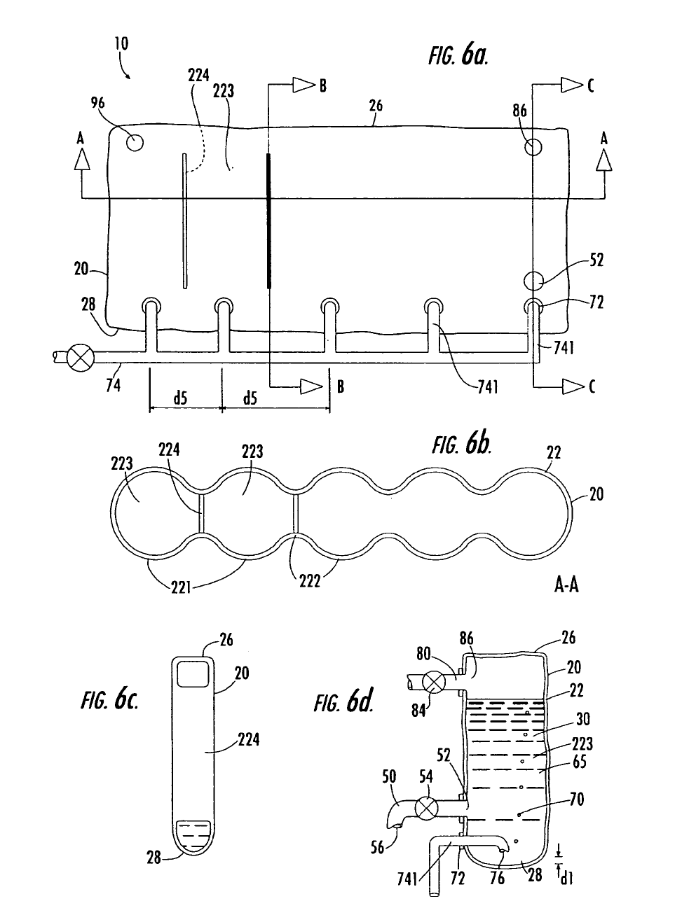

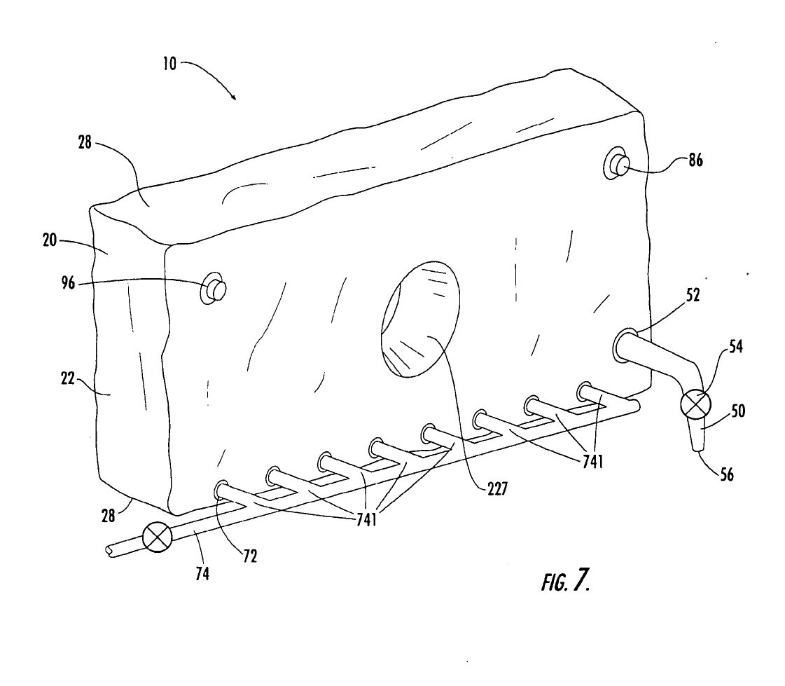

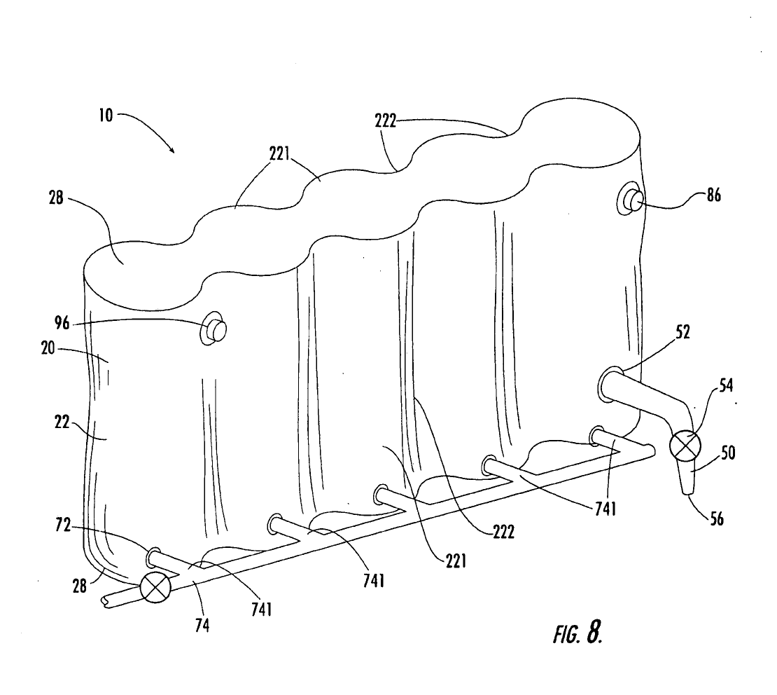

In fourth and fifth embodiments of the present invention, and with reference to FIGS. 5 and 6 respectively, the device (10) also comprises a transparent and/or translucent container (20), having a top end (26) and a bottom end (28). The container (20) comprises a side wall (22) which is preferably substantially rectangular in cross-section, having a large length to width aspect ratio, as shown for the fourth embodiment of the present invention (FIG. 5). Thus, the container (20) of the fourth embodiment is substantially box-like, having typical height-length-width dimensions of 130 cm by 70 cm by 10 cm, respectively. The height to length ratio of the device is typically between, for example, about 1 and about 3, and preferably about 1.85. The height-to-width ratio of the device is typically between 5 and about 30, and preferably about 13.

Alternatively, and as shown in FIG. 6 with respect to the fifth embodiment of the present invention, the sidewall (22) may comprise a substantially accordion-shaped horizontal cross-section, having a series of parallel crests (221) intercalated with troughs (222) along the length of the container (20), thereby defining a series of adjacent chambers (223) in fluid communication with each other. Optionally, the sidewall (22) of the fifth embodiment may further comprise a plurality of vertical webs (224), each internally joining pairs of opposed troughs, thereby separating at least a vertical portion of each chamber (223) from adjacent chambers (223). The webs (224) not only provide increased structural integrity to the container (20), but also effectively separate the container (20) into smaller volumes, providing the advantage of enhanced circulation. In other words, the effectiveness of air bubbles in promoting cell circulation is far higher in smaller enclosed volumes than in a larger equivalent volume. In fact, a proportionately higher volume flow rate for the air bubbles is required for promoting air circulation in a large volume than in a number of smaller volumes having the same combined volume of medium. In the fourth and fifth embodiments, bottom end (28) is substantially semi-cylindrical or may be alternatively convex, substantially flat, or any other suitable shape. In the fourth and fifth embodiments, the container (20) comprises an internal fillable volume (30) which is typically between 10 and 100 liters, though device (10) may alternatively have an internal volume greater than 100 liters, and also greater than 200 liters. Internal volume (30) may be filled with a suitable sterile biological cell and/or tissue culture medium (65) and/or axenic inoculant (60) and/or sterile air and/or required other sterile additives such as antibiotics or fungicides for example, as hereinafter described. In the aforementioned fourth and fifth embodiments, the container (20) is substantially non-rigid, being made preferably from a non-rigid plastics material chosen from the group comprising polyethylene, polycarbonate, a copolymer of polyethylene and nylon, PVC and EVA, for example, and, optionally, the container (20) may be made from a laminate of more than one layer of materials.

As for the first, second and third embodiments, device (10) of the fourth and fifth embodiments is also for aerobic use. In the fourth and fifth embodiments, the container (20) further comprises at least one air inlet for introducing sterile air in the form of bubbles (70) into culture medium (65) through a plurality of air inlet openings (72). In the fourth and fifth embodiments, air inlet comprises at least one air inlet pipe (74) connectable to a suitable air supply (not shown) and in communication with a plurality of secondary inlet pipes (741), each secondary inlet pipe (741) extending from inlet opening (72) to a location inside container (20) at a distance d1 from the bottom of bottom end (28), wherein d1 may be typically around 1 cm, though it could be greater or smaller than 1 cm. The plurality of inlet openings (72), are horizontally spaced one from another by a suitable spacing d5, typically between about 5 cm and about 25 cm, and preferably about 10 cm. The at least one air inlet pipe (74) and secondary inlet pipes (741) may be made from silicon or other suitable plastic material and is preferably flexible. Each of secondary inlet pipes (741) thus comprises an air outlet (76) of suitable diameter to produce air bubbles (70) of a required mean diameter. These bubbles not only aerate the medium (65), but also serve to mix the contents of the container, thereby minimizing sedimentation at the bottom end (28) as well, as hereinbefore described. The size of the bubbles delivered by the air inlet will vary according to the use of the device, ranging from well under 1 mm to over 10 mm in diameter. In some cases, particularly relating to plant cells, small bubbles may actually damage the cell walls, and a mean bubble diameter of not less than 4 mm substantially overcomes this potential problem. In other cases, much smaller bubbles are beneficial, and a sparger may be used at least one of air outlets (76) to reduce the size of the bubbles. In yet other cases air bubbles of diameter 10 mm or even greater may be optimal. Optionally, each outlet (76) may be restrained in position at bottom end (28) by using a tether (not shown) or by another mechanism known in the art.

The fourth and fifth embodiments of the present invention are especially adapted for processing relatively large volumes of inoculant.

In all the aforementioned embodiments, the air inlet optionally comprises a suitable pressure gauge for monitoring the air pressure in the container (20). Preferably, pressure gauge is operatively connected to, or alternatively comprises, a suitable shut-off valve which may be preset to shut off the supply of air to the container (20) if the pressure therein exceeds a predetermined value. Such a system is useful in case of a blockage in the outflow of waste gases, for example, which could otherwise lead to a buildup of pressure inside the container (20). eventually bursting the same.

The container (20) further comprises at least one gas outlet for removing excess air and/or waste gases from container (20). These gases collect at the top end (26) of the container (20). The gas outlet may comprise a pipe (90) having an inlet (96) at or near the top end (26). at a distance d4 from the bottom of the bottom end (28), wherein d4 is typically 90 cm for the first, second and third embodiments, for example. The pipe (90) may be made from silicon or other suitable plastic material and is preferably flexible. Pipe (90) is connectable to a suitable exhaust (not shown) by a known mechanism. The exhaust means further comprises a blocker, such as a suitable one-way valve or filter (typically a 0.2 micro-meter filter), for example, for substantially preventing introduction of contaminants into container via the gas outlet. At least a portion of the top end (26) may be suitably configured to facilitate the collection of waste gases prior to being removed via inlet (96). Thus, in the first and second embodiments, the upper portion of the top end (26) progressively narrows to a minimum cross sectional area near the location of the inlet (96). Alternatively, at least the upper portion of the top end (26) may be correspondingly substantially frustro-conical or convex. In the fourth and fifth embodiments, the top end (26) may be convex, or relatively flat, for example, and the inlet (96) may be conveniently located at or near a horizontal end of the top end (26).

The container (20) further comprises an additive inlet for introducing inoculant and/or culture medium and/or additives into container. In the aforementioned embodiments, the additive inlet comprises a suitable pipe (80) having an outlet (86) preferably at or near the top end (26), at a distance d3 from the bottom of the bottom end (28), wherein d3 for the first embodiment is typically approximately 68 cm, for example. The pipe (80) may be made from silicon or other suitable plastic material and is preferably flexible. Pipe (80) is connectable by a known connector to a suitable sterilized supply of inoculant and/or culture medium and/or additives. The additive inlet further comprises a blocker for substantially preventing introduction of contaminants into container via additive inlet, and comprises, in these embodiments, a suitable one-way valve or filter (84). Typically, the level of contents of the container (20) remains below the level of the outlet (86).

The container (20) further comprises reusable harvester for harvesting at least a desired first portion of the medium containing cells and/or tissue when desired, thereby enabling the device to be used continuously for at least one subsequent culturing cycle. A remaining second portion of medium containing cells and/or tissue serves as inoculant for a next culture and harvest cycle, wherein culture medium and/or required additives provided. The harvester may also be used to introduce the original volume of inoculant into the container, as well as for enabling the harvested material to flow therethrough and out of the container.

In the aforementioned embodiments, the harvester comprises a pipe (50) having an inlet (52) in communication with internal volume (30), and an outlet (56) outside container (20). The pipe (50) may be made from silicon or other suitable plastic material and is preferably flexible. The pipe (50) is of a relatively large diameter, typically about 2 cm, since the harvested cell and/or tissue flow therethrough may contain clumps of cell particles that may clog narrower pipes. Preferably, inlet (52) is located near the bottom end (28) of the container (20), so that only the container contents above inlet (52) are harvested. Thus, at the end of each harvesting cycle, a second portion of medium containing cells and/or tissues automatically remains at the bottom end (28) of the container (20), up to a level below the level (51) of the inlet (52), which is at a distance d2 from the bottom of bottom end (28). Typically but not necessarily, d2 is about 25 cm for the first embodiment.

Optionally and preferably, d2 is selected according to the volume of container (20), such that the portion of medium and cells and/or tissue that remains is the desired fraction of the volume of container (20). Also optionally and preferably, an additional sampling port may be provided (not shown) for removing a sample of the culture media containing cells and/or tissue. The sampling port preferably features an inlet and pipe as for the harvester, and is more preferably located above the harvester. Other port(s) may also optionally be provided.

Alternatively, inlet (52) may be located at the lowest point in the container (20), wherein the operator could optionally manually ensure that a suitable portion of medium containing cells and/or tissue could remain in the container (20) after harvesting a desired portion of medium and cells and/or tissue. Alternatively, all of the medium could optionally be removed. Harvester further comprises flow controller such as a suitable valve (54) and/or an aseptic connector (55) for closing off and for permitting the flow of material into or out of container (20) via harvester. Typically, aseptic connector (55) is made from stainless steel, and many examples thereof are known in the art. Preferably, the harvester further comprises contamination preventer for substantially preventing introduction of contaminants into container via harvester after harvesting.

In the first, second, third; fourth and fifth embodiments, contamination preventer comprises a fluid trap (300). The fluid trap (300) is preferably in the form of a substantially U-shaped hollow tube, one arm of which is mounted to the outlet (56) of the harvester, and the other arm having an external opening (58), as shown for the first embodiment, for example, in FIG. 1(b). Harvested cells/tissue may flow out of the device (10) via harvester, fluid trap (300) and opening (58), to be collected thereafter in a suitable receiving tank as hereinafter described. After harvesting is terminated, air could possibly be introduced into the harvester via opening (56), accompanied by some back-flow of harvested material, thereby potentially introducing contaminants into the device. The U-tube (300) substantially overcomes this potential problem by trapping some harvested material, i.e., cells/tissues, downstream of the opening (56) thereby preventing air, and possible contaminants, from entering the harvester. Once the harvester is closed off via valve (54), the U-tube (300) is removed and typically sterilized for the next use or discarded. The U-tube (300) may be made from stainless steel or other suitable rigid plastic materials. In the aforementioned embodiments, remaining second portion of medium containing cells and/or tissue typically comprises between 10% and 20% of the original volume of culture medium and inoculant, though second portion may be greater than 20%, up to 45% or more, or less than 10%, down to 2.5% or less, of the original volume, if required.

Device (10) optionally further comprises an attacher for attaching same to an overhanging support structure. In the aforementioned embodiments, support structure may comprise a bar (100) (FIGS. 1, 2, 5) or rings (not shown). In the third embodiment, the attacher may comprise a hook (25) preferably integrally attached to the top end (26) of the container (20). Alternatively, and as shown for the first and second embodiments in FIGS. 1 and 2 respectively, the attacher may comprise a preferably flexible and substantially cylindrical loop (27) of suitable material, typically the same material as is used for the container (20), either integral with or suitably attached (via fusion welding, for example) to the top end (26) of the device. Alternatively, and as shown for the fourth embodiment in FIG. 5, attacher may comprise a preferably flexible and substantially cylindrical aperture (227) made in the sidewall (22) of container (20), extending through the depth thereof. The fifth embodiment may optionally be supported by a series of hooks (not shown) integrally or suitably attached preferably to the top end (26) of the device (10).

Optionally, the containers may be supported in a suitable support jacket. For example, in the fourth embodiment, the device (10) may be supported in a support jacket consisting of a suitable outer support structure comprising an internal volume sized and shaped to complement the datum external geometry of at least the sidewall (22) and bottom end (28) of the device when nominally inflated. The outer support structure may be substantially continuous, with openings to allow access to the inlets and outlets to the device (10), and further has a suitable door or opening either at the side, top or bottom to allow a device (10) to be inserted into the support jacket or removed therefrom. The datum geometry of the device may be defined as the shape of the device (10) when it is inflated to its design capacity. At this point, its shape is nominally is design shape, and therefore its internal volume is nominally its design volumetric capacity. However, when such a device comprising flexible walls is actually filled with a liquid medium, the geometry of the device tends to deviate from the datum geometry, tending to bulge preferentially at the bottom the device where the pressure is greatest, and increasing stresses in the wall material considerably. A support jacket as described for example and having the required structural attributes also helps in maintaining the geometry of the device, and reduces the wall stresses, minimizing risk of rupture of the sidewall (22), for example and thereby ensuring a longer working life for each device.

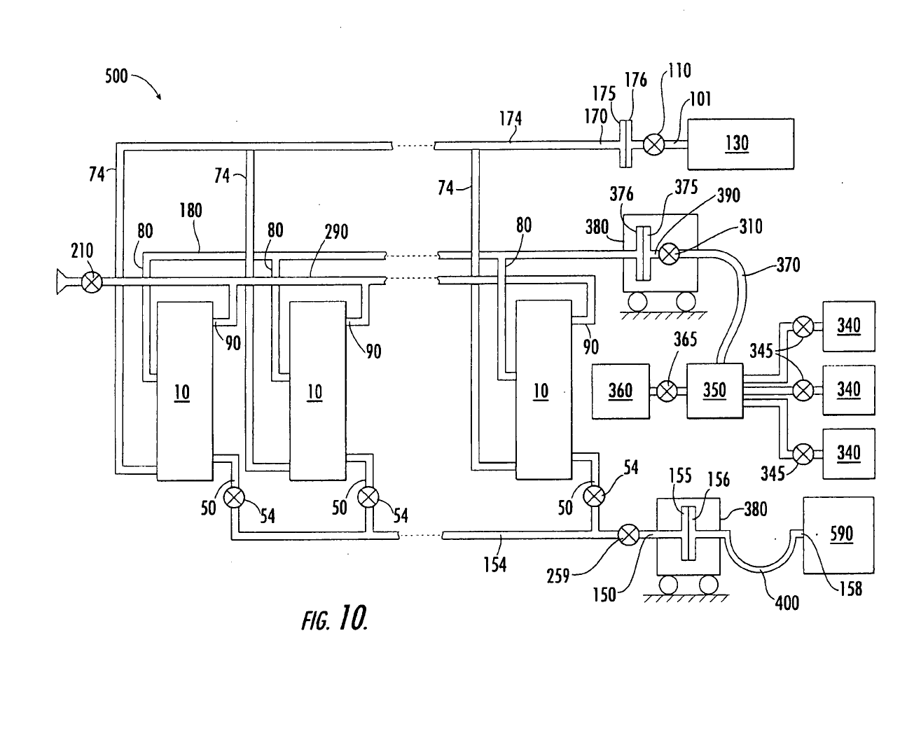

Alternatively, the containers may be supported in a suitable support structure. For example, in the fourth and fifth embodiments of the present invention, the device (10) may be supported in a support structure (400) comprising a pair of opposed frames (405), (406), as illustrated, for example, in FIG. 9. Each frame (405), (406) is typically rectangular comprising substantially parallel and horizontal upper and lower load-carrying members (410) and (420) respectively, spaced by a plurality of substantially parallel vertical support members (430), at least at each longitudinal extremity of the load-carrying members (410), (420), and integrally or otherwise suitably joined to the upper and lower load-carrying members, (410) and (420) respectively. The lower support member (420) of each frame (405) and (406) comprises suitably shaped lower supports adapted for receiving and supporting a corresponding portion of the bottom end (28) of the containers (20). Typically, the lower supports may take the form of a suitably shaped platform projecting from each of the lower support-members (420) in the direction of the opposed frame. Alternatively, the lower supports may take the form of a plurality of suitably shaped tabs (460) projecting from each of the lower support members (420) in the direction of the opposed frame. The frames (405), (406) are spaced from each other by strategically located spacing bars (450), such that the container (20) may be removed relatively easily from the support structure (400) and a new container (20) maneuvered into place, i.e., without the need to dismantle the support frame (400). The spacing bars (450) may be integrally connected to the frames (405), (406), as by welding for example. Preferably, though, the spacing bars (450) are releasably connected to the frames (405), (406), such that the frames (405), (406) may be separated one from the other, and also permitting the use of different sized spacing bars to connect the frames (405), (406), thereby enabling the support structure (400) to be used with a range of containers (20) having different widths. Optionally, and preferably, the frames (405). (406) each comprise at least one interpartitioner (470). interpartitioner (470) may take the form of a vertical web projecting from each frame (405), (406) in the direction of the opposed frame, and serves to push against the sidewall (22) at a predetermined position, such that opposed pairs of interpartitioner (470) effectively reduce the width of the container (20) at the predetermined position, thereby creating, between adjacent opposed pairs of interpartitioner (470), for example, a partitioning or semi partitioning of the internal space (30) of the container (20). Thus, the interpartitioner (470) may typically deform the sidewall (22) of a container (20) according to the fourth embodiment (see FIG. 5) to a shape resembling that of the sidewall (22) of the fifth embodiment (see FIG. 6). Of course, when used with a container (20) according to the fifth embodiment of the present invention, the interpartitioner (470) are located on the frames (405), (406) such as to engage with the troughs (222) of the sidewall (22), and thus particularly useful in maintaining the shape of the containers (20). Thus, adjacent partitioner (470) on each frame are spaced advantageously spaced a distance (d5) one from another. Preferably, interpartitioner (470) comprise suitable substantially vertical members (472) spaced from the upper and lower support members, (410), (420), in a direction towards the opposed frame with suitable upper and lower struts (476), (474) respectively. The support structure F(400) thus not only provides structural support for the containers (20), particularly of the fourth and fifth embodiments, it also provides many open spaces between each of the load carrying members for enabling each of the air inlet, the gas outlet, the harvester and the additive inlet to pass therethrough. Optionally, support structure (400) may comprise rollers or castors (480) for easing transportation of the containers (20) within a factory environment, for example.

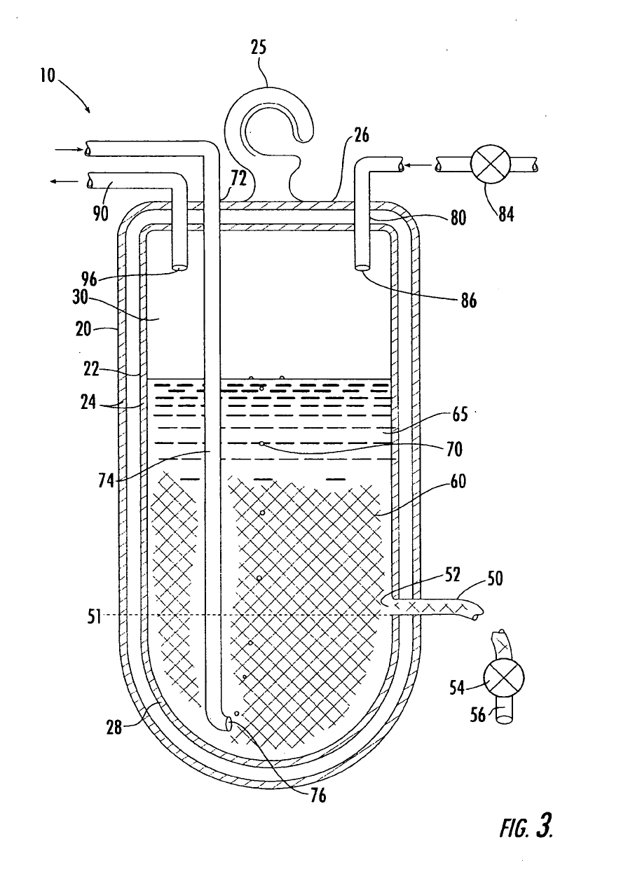

The container (20) may optionally be formed by fusion bonding two suitable sheets of suitable material, as hereinbefore exampled, along predetermined seams. Referring to the first and second embodiments for example, two sheets (200) of material may be cut in an approximately elongated rectangular shape and superposed one over the other, FIG. 4. The sheets are then fusion bonded together in a manner well known in the art to form seams along the peripheries (205) and (206) of the two longer sides, and along the periphery of one of the shorter ends (210), and again parallel and inwardly displaced thereto to form a seam (220) at the upper end of the container (20). The fusion weld seams (207) and (208) along the long sides and situated between these parallel short end seams (210) and (220) may be cut off or otherwise removed, effectively leaving a loop of material (27). The bottom end (28) of the container (20) is formed by fusion bonding the remaining short end of the sheets along two sloping seam lines, (230) and (240), mutually converging from the seams (205) and (206) of the long sides. Optionally, the two sloping seam lines (230) and (240) may be joined above the apex by another fusion welded seam line (260) approximately orthogonal to the long side seams (205) and (206). Prior to fusion welding the two sheets together, rigid plastic bosses (270), (290), (280) and (250) may be fusion welded at locations corresponding to the air inlet, gas outlet, additive inlet and harvester, respectively. These bosses provide suitable mechanical attachment points for each of the corresponding input(s) and output(s). The third, fourth and fifth embodiments of the present invention may be manufactured in a similar manner to the first and second embodiments, substantially as described above, mutatis mutandis.

In all embodiments, the device (10) is made from a material or materials that are biologically compatible and which enable the container to be sterilized prior to first use.

### EXAMPLE 2

### Illustrative System

The present invention also relates to a battery of disposable devices for axenically culturing and harvesting cells and/or tissue in cycles, wherein each of a plurality of these devices is structurally and operationally similar to device (10), hereinbefore defined and described with reference to the first through the fifth embodiments thereof.

Referring to FIG. 10, a battery (500) comprises a plurality of devices (10), as hereinbefore described with respect to any one of the first through the fifth embodiments, which are held on a frame or frames (not shown) with an attacher or support structure (400), for example. Typically, the battery (500) may be divided into a number of groups, each group comprising a number of devices (10).

In the preferred embodiment of the battery (500), the air inlets of the devices (10) in each group are interconnected. Thus the air inlet pipes (74) of each device (10) of the group are connected to common piping (174) having a free end (170), which is provided with an aseptic connector (175). Sterilized air is provided by a suitable air compressor (130) having a suitable sterilizer or blocker (110) such as one or more filters. The compressor (130) comprises a delivery pipe (101) having an aseptic connector (176) at its free end which is typically connectable to the aseptic connector (175) located at the free end of common piping (174). This connection is made at the beginning of each run of growth/harvesting cycles in a mobile sterile hood (380) to ensure that sterile conditions are maintained during the connection. The sterile hood (380) provides a simple relatively low-cost system for connecting the various services, such as air, media, inoculant and harvested cells, to and from the group of devices (10) under substantially sterile conditions. Similarly, at the end of each run of growth/harvesting cycles, the connectors (175) and (176) are disconnected in the sterile hood (380), and the used devices are discarded, allowing the connector (175) at the compressor end to be connected to the connector (176) of a new group of devices. Sterilized air is typically provided continuously, or alternatively in predetermined pulses, during each culturing cycle.

In the preferred embodiment of the battery (500), excess air and/or waste gases from each of the devices (10) is removed to the atmosphere via common piping (290) suitably connected to each corresponding gas outlet (90). Common piping (290) is provided with a suitable contaminant preventer (210), such as one or more filters, for preventing contaminants from flowing into devices (10). Alternatively, the gas outlet (90) of each device (10) may be individually allowed to vent to the atmosphere, preferably via suitable filters which substantially prevent contaminants from flowing into the device (10).

Media and additives are contained in one or more holding tanks (340). For example, micro elements, macro elements and vitamins may be held in different tanks, while additives such as antibiotics and fungicides may also held in yet other separate tanks. A pumper (345) serving each tank enable the desired relative proportions of each component of the media and/or additives to be delivered at a predetermined and controllable flow rate to a static mixer (350), through which water—either distilled or suitably filtered and purified—flows from a suitable supply (360), preferably with the aid of a suitable pumper (365) (FIG. 10). By adjusting the flow rates of pumpers (345) and (365), for example, the concentration of media as well as additives available to be delivered into devices (10) may be controlled. Media and/or additives mixed with water may then be delivered from the static mixer (350) under sterile conditions via a filter (310) and a delivery pipe (370) having an aseptic connector (375) as its free end (390).

In the preferred embodiment of the battery (500), the inlet of additive pipe (80) of each corresponding device (10) in the group of devices, are interconnected via common piping (180), which comprises at its free end a common aseptic connector (376), common aseptic connector (376) may then be connected, in the sterile hood (380), to the aseptic connector (375) at the free end (390) of the media and additive pipe (370), thus enabling each device (10) of the battery, or of the group, to be supplied with media and additives. At the end of the life of the devices (10), and prior to discarding the same, the aseptic connectors (375) and (376) are disconnected n the sterile hood. The aseptic connector (375) is then ready to be connected to the new aseptic connector (376) of the next sterilized group of new devices (10) of the battery, ready for the next run of culturing/harvesting cycles.

The sterile hood (380) may also optionally be used for connecting the media/additives tank (350) to each one of a number of groups of devices (10) in the battery, in turn, during the useful lives of the devices in these groups. Thus, when one group of devices has been serviced with media/additives, the aseptic connector (376) of this group is aseptically sealed temporarily in the sterile hood (380), which is then moved to the next group of devices where their common aseptic connector (376) is connected to the sterile connector (375) of the pipe (370), thus enabling this group of devices to be serviced with media/additives.

In a different embodiment of the battery (500), a mobile sterile hood (380) may be used to connect together the free end (390) of a preferably flexible delivery pipe connected to static mixing tank (350), to the additive inlet of each device (10) in turn. The sterile hood (380) may then be moved from one device (10) to the next, each time the end (390) being connected to the inlet end of the corresponding pipe (80) to enable media to be provided to each device in turn. The sterile hood (380), together with aseptic connector, preferably made from stainless steel, at end (390) and the inlet of the pipe (80) of the corresponding device (10), respectively, enable each device (10) to be easily connected and subsequently disconnected to the end (390) and thus to the media supply, under sterile conditions. Many other examples of suitable connector for connecting two pipes together are well known in the art. Suitable filters are provided at the end (390) and at the pipe (80), respectively, to prevent or at least minimize potential contamination of the container contents. The sterile hood (380) may thus be automatically or manually moved from device (10) to device (10), and at each device in turn, an operator may connect the device (10) to the media supply using the sterile hood (380), fill the device with a suitable quantity of media and/or additives, and subsequently disconnect the sterile hood (380) from the device, to then move on to the next device. Of course, the end (390) may be adapted to comprise a plurality of connector (375) rather than just a single sterilized connector (375), so that rather than one, a similar plurality of devices (10) having corresponding connector (376) may be connected at a time to the media supply via the trolley (380).

Each time, prior to connecting end (390) to each device or set or group of devices, the corresponding connectors (375) and (376) are typically sterilized, for example through an autoclave.

In yet another embodiment of the battery (500), a single pipe or a set of pipes (not shown) connect static mixer (350), to one device (10) or to a corresponding set of devices (10), respectively, at a time, wherein a conveyor system transports the device (10) or set of devices (10) to the single pipe or set of pipes, respectively, or vice versa. After filling the device (10) or set of devices (10), the conveyor enables a further device (10), or a further set of devices (10) to be connected to the static mixer (350) through the single pipe or set of pipes, respectively.

In the preferred embodiment of the battery (500), the harvesters of each of the devices (10) of the group are interconnected. Thus the harvesting pipes (50) of each device (10) are connected to common harvesting piping (154) having a free end (150), which is provided with an aseptic connector (155). Preferably, each of the harvesting pipes (50) may comprise a valve (54), as hereinbefore described, to close off or permit the flow of harvested cells from each corresponding device (10). Thus, for example, if it is determined that a number of devices in a particular group are contaminated, while the other devices are not, then the cells in these latter devices may be harvested without fear of contamination from the former devices, so long as the valves (54) of the contaminated devices remain closed. Preferably, common piping further comprises a common shut-off valve (259) upstream of the aseptic connector (155). Preferably, contamination preventer is provided for substantially preventing introduction of contaminants into container via harvester after harvesting.

In the preferred embodiment, the contamination preventer comprises a substantially U-shaped fluid trap (400), having an aseptic connector (156) at one arm thereof, the other arm having an opening (158) in fluid communication with a receiving tank (590). The aseptic connectors (155) and (156) are then interconnected in the mobile sterile hood (380) under sterile conditions. Harvesting is then effected by opening the valves (54) of all the devices in the group which are not contaminated, as well as common valve (259). Cells from the group will then flow into the receiving tank (590), preferably under gravity, though in some cases a suitable pump may be used. After harvesting is completed, the aseptic connectors (155) and (156) may be disconnected in the sterile hood (380), which can then be moved to the next group of devices (10): the corresponding aseptic connector (155) of this group may then be interconnected with aseptic connector (156) of the U-tube (400), and thereby enable the cells of this group of devices to be harvested.

In another embodiment of the battery (500), a single pipe or a set of pipes (not shown) may connect common receiving tank to a device (10) or a corresponding set of devices (10), respectively, at a time, wherein a conveyor system transports the device (10) or set of devices (10) to the single pipe or set of pipes, respectively, or vice versa. After harvesting the device (10) or set of devices (10), the conveyor enables a further device (10) or set of devices (10) to be connected to the- common receiving tank through a single pipe or set of pipes, respectively.

In another embodiment of the battery (500), each device (10) may be individually harvested, wherein the harvester of each device comprises a contamination preventer for substantially preventing introduction of contaminants into container via harvester after harvesting. In this embodiment, the contamination preventer comprises U-shaped fluid trap (400) as hereinbefore described, having an aseptic connector (156) at one arm thereof, the other arm having an opening (158) in fluid communication with a receiving tank (590). The harvester comprises an aseptic connector (55) which may be connected to the aseptic connector (156) of the fluid trap (400) in the mobile sterile hood (380) under sterile conditions. Harvesting is then effected by opening the valve (54) of the device, wherein cells will then flow into the receiving tank, preferably under gravity, though in some cases a suitable pump may be used. After harvesting is completed, these aseptic connectors, (55) and (156), may be disconnected in the sterile hood (380), which can then be moved to the next device (10): the corresponding aseptic connector (55) of the harvester of this device may then be interconnected with aseptic connector (156) of the U-tube (400), and thereby enable the cells of this next device to be harvested.

In the preferred embodiment of the battery (500), the harvester may also be used for initially providing inoculant at the start of a new run of growth/harvesting cycles. Thus, inoculant may be mixed with sterilized medium in a suitable tank having a delivery pipe comprising at its free end an aseptic connector which is connected to the aseptic connector (155) of the common harvesting piping (154) in the sterile hood (380). Inoculant may then be allowed to flow under gravity, or with the aid of a suitable pump, to each of the devices (10) of the group via common harvesting piping (154), after which the aseptic connectors are disconnected in the sterile hood.

Alternatively, the inoculant may be introduced into the devices via the additive inlet, in particular the additive common piping (180), in a similar manner to that hereinbefore described regarding the harvester and the common harvesting piping (155), mutatis mutandis.

According to preferred embodiments of the present invention, the operation of the previously described individual device and/or battery is controlled by a computer (600), as shown with regard to FIG. 1C. The computer is optionally and preferably able to control such parameters of the operation of the battery and/or of a device according to the present invention as one or more of temperature, amount and timing of gas or gas combination entering the container, amount and timing of gas being allowed to exit the container, amount and timing of the addition of at least one material (such as nutrients, culture medium and so forth), and/or amount of light. The computer may optionally also be able to detect the amount of waste being produced.

The computer is preferably connected to the various measuring instruments present with regard to the operation of the present invention, as an example of a system for automating or semi-automating the operation of the present invention. For example, the computer (600) is preferably connected to a gauge (602) or gauges for controlling the flow of a gas or gas combination. Gauge (602) is preferably connected to a pipe (74) connectable to a suitable air supply (604), and controls the flow of air or other gas(es) to pipe (74).

The computer (600) is also preferably connected to a temperature gauge (606), which is more preferably present in the environment of container (20) but more preferably not within container (20). The computer (600) is also optionally and preferably able to control a mechanism for controlling the temperature (608), such as a heater and/or cooler for example.

The computer (600) is optionally and preferably connected to a gauge (610) for controlling the flow of media and/or other nutrients from a nutrient/media container (612; hereinafter referred to collectively as a nutrient container) to container (20) through pipe (80) of the present invention. Computer (600) may also optionally, additionally or alternatively, control valve (84). Also optionally, only one of valve (84) or gauge (610) is present.

The computer (600) is preferably connected to at least one port of the container, and more preferably (as shown) is connected at least to a harvest port (shown as pipe (50)) and optionally as shown to a sample port (612). Optionally, the sample port and the harvest port may be combined. The computer optionally may control an automated sampler and/or harvester for removing portions of the contents of the container, for testing and/or harvesting (not shown). The computer may also optionally be connected to an analyzer (614) for analyzing these portions of contents, for example in order to provide feedback for operation of the computer.

### EXAMPLE 3

### Illustrative Plant Cell Culturing Method

The present invention also relates to a method for culturing and harvesting plant cells in a multiple-use disposable device. The device is optionally and preferably configured according to the device and/or system of Examples 1 and 2 above. In this method, plant cells are preferably placed in a container of the device according to the present invention. This container is preferably constructed of plastic, which may optionally be translucent and/or transparent, and which optionally may be rigid or flexible, or may optionally have a degree of rigidity between rigid and flexible (e.g. semi-rigid for example). Any other additional material(s) are then provided, such as sterile gas or a gas combination, and/or a sterile liquid or a liquid combination, or any other suitable additive. Preferably, the device is constructed to feature a reusable harvester, such that material (plant cells and/or one of the previously described additional materials) may be removed while still permitting at least one additional cell culturing/harvesting cycle to be performed. Optionally and more preferably, the plant cells are cultured in suspension.

According to preferred embodiments of the present invention, the plant cells are cultured in suspension in a liquid medium, with at least one sterile gas or gas combination (plurality of gases) added as required. Optionally and preferably, the sterile gas comprises a sterile gas combination which more preferably comprises sterile air. The sterile gas and/or gas combination is preferably added to the container through an air inlet during each cycle, either continuously or in pulses, as previously described.

Sterile culture medium and/or sterile additives are preferably placed in the container through an additive inlet as previously described.

The plant cells (as an example of an axenic inoculant) are optionally and preferably added through the harvester. Optionally and preferably, the plant cells in the container are exposed to light, for example through an external light (a source of illumination external to the container), particularly if the container is transparent and/or translucent.

The cells are allowed to grow to a desired yield of cells and/or the material produced by the cells, such as a protein for example.

According to preferred embodiments, excess air and/or waste gases are preferably allowed to leave the container through a gas outlet, optionally and more preferably continuously and/or intermittently.

Also optionally and preferably, the material in the container (such as the cell culture medium for example) is checked for one or more contaminants and/or the quality of the cells and/or cell product(s) which are produced in container. More preferably if one or more contaminants are found to be present or the cells and/or cell product(s) which are produced are of poor quality, the device and its contents are disposed of

At an appropriate time, particularly if contaminant(s) and/or poor quality cells and/or cell product(s) are not found, at least a first portion of the material in the container is preferably harvested, such as medium containing cells and/or cell product(s). More preferably, a remaining second portion of material, such as medium containing cells and/or cell product(s) is allowed to remain in the container, wherein this second portion may optionally serve as inoculant for a next culture/harvest cycle. Next, sterile culture medium and/or sterile additives are provided for the next culture/harvest cycle through the additive inlet.

The previously described cycle is optionally performed more than once. Also, the previously described cycle may optionally be performed with a battery (system) of devices as described with regard to Example 2. Optionally and preferably, the method permits cells to be cultured and/or harvested anaerobically.

For the anaerobic embodiment, a battery (500) of at least one group of devices (10) is provided, wherein the devices do not comprise an air inlet. For at least one device (10) thereof the following process is performed. An axenic inoculant to device via common harvesting piping. Next, sterile culture medium and/or sterile additives is added to the device via common additive inlet piping. Optionally, the device is illuminated as previously described.

The cells in the device are allowed to grow in medium to a desired yield of cells and/or product(s) of the cells. Optionally and preferably, excess air and/or waste gases are permitted to leave the device, more preferably continuously, via common gas outlet piping.

As for the previous method, the material in the container is checked for the presence of one or more contaminant(s) and/or poor quality cells and/or poor quality cell product(s), in which case the container and its contents are preferably disposed of. Also as for the previous method, the cells and/or cell product(s) are preferably harvested at a suitable time, for example when a desired amount of cell product(s) has been produced.

The above method may also optionally be performed aerobically in a battery of disposable devices, such that sterile gas and/or combination of gases, such as sterile air, is provided to device via common air inlet piping.

Typically, a water purification system supplies deionised and pyrogen free water to a tank comprising concentrated media, and diluted media is then pumped to the device (10) via additive inlet. Filters, typically 0.2 micro-meter, are installed in the feed pipes and also just upstream of the additive inlet to minimize risk of contamination of the container contents in each device (10). Alternatively or additionally, a one-way valve may be also be used to minimize this risk.

For the first culturing cycle of each device (10), inoculant, typically a sample of the type of cell that it is required to harvest in the device (10), is premixed with media or water in a steam sterilized container and is introduced into the device (10) via the harvester. Media is then introduced into the device (10) via additive input. For subsequent cycles, only media and/or additives are introduced, as hereinbefore described.

Typically, an air compressor provides substantially sterilized air to each device (10), via a number of filters: a coarse filter for removing particles, a dryer and humidity filter for removing humidity and a fine filter, typically 0.2 micro-meter, for removing contaminants. Preferably, another filter just upstream of the air inlet further minimizes the risk of contamination of the container contents.

For each device (10), all connections to the container (20), i.e., to air inlet, to additive inlet, and preferably also to the gas outlet and to the harvester are autoclave sterilized prior to use, and sterility is maintained during connection to peripheral equipment, including, for example, air supply and exhaust by performing the connections in the sterile hood as hereinbefore described.

Temperature control for each device (10) is preferably provided by a suitable air conditioner. Optional illumination of the device may be provided by suitable fluorescent lights suitably arranged around the device (10), when required for cell growth.

During each culturing cycle of each device (10), the contents of each corresponding container (20) are typically aerated and mixed for about 7 to about 14 days, or longer, under controlled temperature and lighting conditions.

At the end of the culturing cycle for each device (10), the corresponding harvester is typically connected to a presterilised environment with suitable connectors which are sterilized prior and during connection, as hereinbefore described. Harvesting is then effected, leaving behind between about 2.5% to about 45%, though typically between about 10% to about 20%, of cells and/or tissue to serve as inoculant for the next cycle.

The harvested cells/tissues and/or cell product(s) may then optionally be dried, or extracted, as required

According to preferred embodiments of the present invention, the process of cell culturing may optionally be adjusted according to one or more of the following. These adjustments are preferably performed for culturing plant cells. According to a first adjustment, for cells being grown in suspension in culture media, the amount of media being initially placed in the container (e.g. on day zero) is preferably at least about 125% of the recommended amount, and more preferably up to about 200% of the recommended amount of media.

Another optional but preferred adjustment is the addition of media during growth of the cells but before harvesting. More preferably, such media is added on day 3 or 4 after starting the culture process. Optionally and more preferably, the media comprises concentrated culture media, concentrated from about 1 to about 10 times and thereby providing a higher concentration of nutrients. It should be noted that preferably a sufficient medium is provided that is more preferably at a concentration of at least about 125% of a normal concentration of medium. Addition of media means that fresh media is added to existing media in the container. When added as a concentrated solution, preferably the resultant media concentration is close to the normal or initial concentration. Alternatively, the media in the container may optionally be completely replaced with fresh media during growth, again more preferably on day 3 or 4 after starting the culture process.

Another optional but preferred adjustment is the use of higher sucrose levels than is normally recommended for plant cell culture, for example by adding sucrose, such that the concentration in the media may optionally be 40 g/l rather than 30 g/l. One or more other sugars may optionally be added, such as glucose, fructose or other sugars, to complement sucrose. Sucrose (and/or one or more other sugars) is also optionally and preferably added during the cell culture process, more preferably on day 3 or 4 after starting the culture process.

Another optional adjustment is the addition of pure oxygen during the cell culture process, more preferably on day 3 or 4 after starting the culture process.

Another optional adjustment is the use of increased aeration (gas exchange), which as shown in greater detail below, also results in an increased cell growth rate in the device according to the present invention.

### EXAMPLE 4

### Experimental Example with Vinca Rosea Cells

This experiment was performed with cells from Vinca rosea also known as rose periwinkle.

A group of 10 bioreactors (each a device according to the invention), each with a container made from polyethylene-nylon copolymer, (0.1 mm wall thickness, 20 cm diameter, 1.2 m height), complete with 30 mm ports at 5 cm (for air inlet), 25 cm (for harvester), 68 cm (additive inlet), and 90 cm (gas outlet) from the bottom, effective fillable volume about 10 liters was used. The bioreactors, together with their fittings, were sterilized by gamma irradiation (2.5 mRad).

Nine liters of Schenk & Hildebrandt mineral/vitamin medium, 2 mg/l each of chlorophenoxyacetic acid and 2,4-dichlorophenoxyacetic acid, 0.2 mg/l kinetin, 3% sucrose, and 900 ml packed volume initial inoculum of line V24 Catharanthus roseus (Vinca) cells were introduced into each bioreactor. The volume of air above the surface of the medium was 3 L. Aeration was carried out using a flow volume of 1.5 l/min sterile air, provided through a 4 mm orifice (air inlet), located 1 cm from the bottom of the container.

The bioreactors were mounted in a controlled temperature room (25° C.) and culturing was continued for 10 days, until the packed volume increased to about 7.5 l (75% of the total volume; a doubling rate of 2 days during the logarithmic phase). At this time point, cells were harvested by withdrawing 9 liters of medium and cells through the harvester and 9 liters of fresh sterile medium together with the same additives were added via the additive inlet. Cells were again harvested as above at 10-day intervals, for 6 additional cycles, at which time the run was completed.

A total weight of 6.5 kg fresh cells (0.5 kg dry weight) was thus collected over various periods of time, such as seven, ten or fourteen day intervals, from each of the 10 l capacity bioreactors. These cells had a 0.6% content of total alkaloids, the same as the starting line. Therefore, clearly the device of the present invention was able to maintain and grow the cells in culture in a healthy and productive state, while maintaining similar or identical cell characteristics as for cells from the starting line.

### EXAMPLE 5

### Experimental Example with Plant Cells

This Example provides a description of experiments that were performed with transformed plant cells, cultured in the device of the present invention, according to the method of the present invention.

Experimental Procedures:

Plasmid vectors
CE-T—Was constructed from plasmid CE obtained from Prof. Galili [U.S. Pat. No. 5,367,110 Nov. 22, (1994)].

Plasmid CE was digested with SalI.

The SalI cohesive end was made blunt-ended using the large fragment of DNA polymerase I. Then the plasmid was digested with PstI and ligated to a DNA fragment coding for the ER targeting signal from the basic endochitinase gene [ Arabidopsis thaliana] ATGAAGACTAATCTTTTTCTCTTTCTCATCTTTTCA

CTTCTCCTATCATTATCCTCGGCCGAATTC, and vacuolar targeting signal from Tobacco chitinase A: GATCTTTTAGTCGATACTATG digested with SmaI and PstI.

The SalI cohesive end was made blunt-ended using the large fragment of DNA polymerase I. Then the plasmid was digested with PstI and ligated to a DNA fragment coding for the ER targeting signal (SEQ ID NO: 1), a non relevant gene, and vacuolar targeting signal (SEQ ID NO: 2), digested with SmaI and PstI.
pGREENII—obtained from Dr. P. Mullineaux [Roger P. Hellens et al., (2000) Plant Mol. Bio. 42:819-832]. Expression from the pGREEN II vector is controlled by the 35S promoter from Cauliflower Mosaic Virus, the TMV (Tobacco Mosaic Virus) omega translational enhancer element and the octopine synthase terminator sequence from Agrobacterium tumefaciens.

cDNA

hGCD—obtained from ATCC (Accession No. 65696), GC-2.2 [GCS-2 kb; lambda-EZZ-gamma3 Homo sapiens] containing glucosidase beta acid [glucocerebrosidase]. Insert lengths (kb): 2.20; Tissue: fibroblast WI-38 cell.

Construction of Expression Plasmid

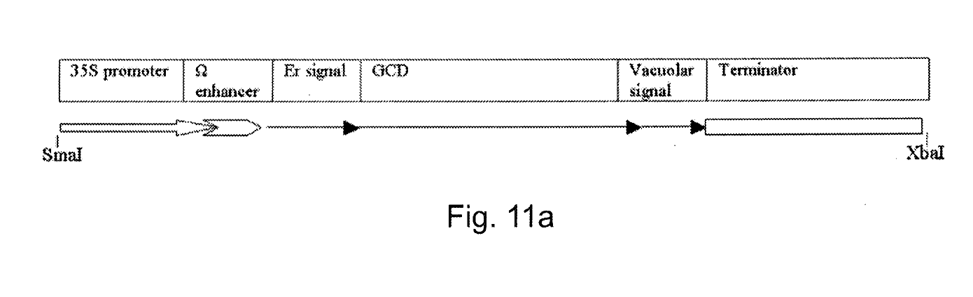

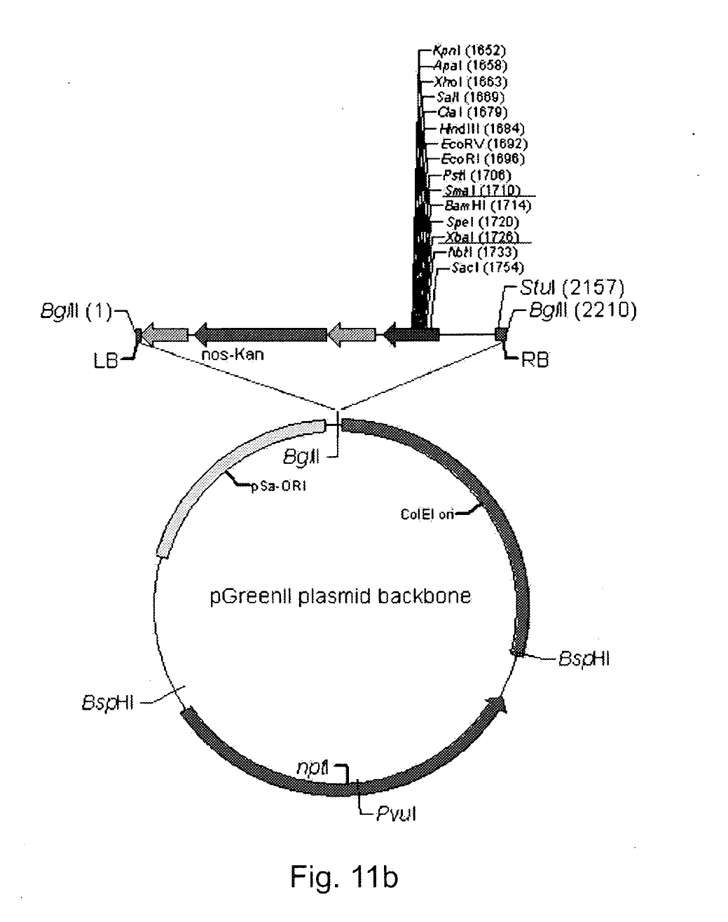

The cDNA coding for hGCD (ATTC clone number 65696) was amplified using the forward: 5′ CAGAATTCGCCCGCCCCTGCA 3′ and the reverse: 5′ CTCAGATCTTGGCGATGCCACA 3′ primers. The purified PCR DNA product was digested with endonucleases EcoRI and BglII (see recognition sequences underlined in the primers) and ligated into an intermediate vector having an expression cassette E-T digested with the same enzymes. The expression cassette was cut and eluted from the intermediate vector and ligated into the binary vector pGREENII using restriction enzymes SmaI and XbaI, forming the final expression vector. Kanamycine resistance is conferred by the NPTII gene driven by the nos promoter obtained together with the pGREEN vector (FIG. 11B). The resulting expression cassette is presented by FIG. 11A.

The resulting plasmid was sequenced to ensure correct in-frame fusion of the signals using the following sequencing primers: 5′ 35S promoter: 5′ CTCAGAAGACCAGAGGGC 3′, and the 3′ terminator: 5′ CAAAGCGGCCATCGTGC 3′.

Establishment of Carrot Callus and Cell Suspension Culture

Establishment of carrot callus and cell suspension cultures were performed as described previously by Torres K. C. (Tissue culture techniques for horticular crops, p.p. 111, 169).

Transformation of Carrot Cells and Isolation of Transformed Cells.

Transformation of carrot cells was preformed using Agrobacterium transformation by an adaptation of a method described previously [Wurtele, E. S. and Bulka, K. Plant Sci. 61:253-262 (1989)]. Cells growing in liquid media were used throughout the process instead of calli. Incubation and growth times were adapted for transformation of cells in liquid culture. Briefly, Agrobacteria were transformed with the pGREEN II vector by electroporation [den Dulk-Ra, A. and Hooykaas, P. J. (1995) Methods Mol. Biol. 55:63-72] and then selected using 30 mg/ml paromomycine antibiotic. Carrot cells were transformed with Agrobacteria and selected using 60 mg/ml of paromomycine antibiotics in liquid media.

Screening of Transformed Carrot Cells for Isolation of Calli Expressing High Levels of GCD

14 days following transformation, cells from culture were plated on solid media at dilution of 3% packed cell volume for the formation of calli from individual clusters of cells. When individual calli reached 1-2 cm in diameter, the cells were homogenized in SDS sample buffer and the resulting protein extracts were separated on SDS-PAGE [Laemmli U., (1970) Nature 227:680-685] and transferred to nitrocellulose membrane (hybond C nitrocellulose, 0.45 micron. Catalog No: RPN203C From Amersham Life Science) as described in greater detail below. Western blot for detection of GCD was preformed using polyclonal anti hGCD antibodies (described herein below). Calli expressing significant levels of GCD were expanded and transferred to growth in liquid media for scale up, protein purification and analysis.

Upscale Culture Growth in a Device According to the Present Invention

An about 1 cm callus of genetically modified carrot cells containing the rh-GCD gene was plated onto Murashige and Skoog (MS) 9 cm diameter agar medium plate containing 4.4 gr/l MSD medium (Duchefa), 9.9 mg/l thiamin HCI (Duchefa), 0.5 mg folic acid (Sigma) 0.5 mg/l biotin (Duchefa), 0.8 g/l Casein hydrolisate (Duchefa), sugar 30 g/l and hormones 2-4 D (Sigma). The callus was grown for 14 days at 25° C.

Suspension cell culture was prepared by sub-culturing the transformed callus in a MSD (Murashige & Skoog (1962) containing 0.2 mg/l 2,4-dicloroacetic acid) liquid medium, as is well known in the art. The suspension cells were cultivated in 250 ml Erlenmeyer flask (working volume starts with 25 ml and after 7 days increases to 50 ml) at 25° C. with shaking speed of 60 rpm. Subsequently, cell culture volume was increased to 1 L Erlenmeyer by addition of working volume up to 300 ml under the same conditions. Inoculum of the small bio-reactor (10 L) [see WO 98/13469] containing 4 L MSD medium, was obtained by addition of 400 ml suspension cells derived from two 1 L Erlenmeyer that were cultivated for seven days. After week of cultivation at 25° C. with 1 L pm airflow, MSD medium was added up to 10 L and the cultivation continued under the same conditions. After additional five days of cultivation, most of the cells were harvested and collected by passing the cell media through 80μ net. The extra medium was squeezed out and the packed cell cake was store at −70° C.

In a first experiment, growth of transformed (Glucocerebrosidase (GCD)) carrot cell suspension was measured in a device according to the present invention as opposed to an Erlenmeyer flask. Growth was measured as peck cell volume (4000 rpm) and as dry weight. Measuring growth in the Erlenmeyer flask was performed by starting 21 flasks and harvesting 3 flasks every day. The harvested flasks were measured for wet weight, dry weight and GCD content. Reactor harvest was performed by using the harvest port (harvester); each day 50 ml of suspension were harvested for wet and dry weight measurement.

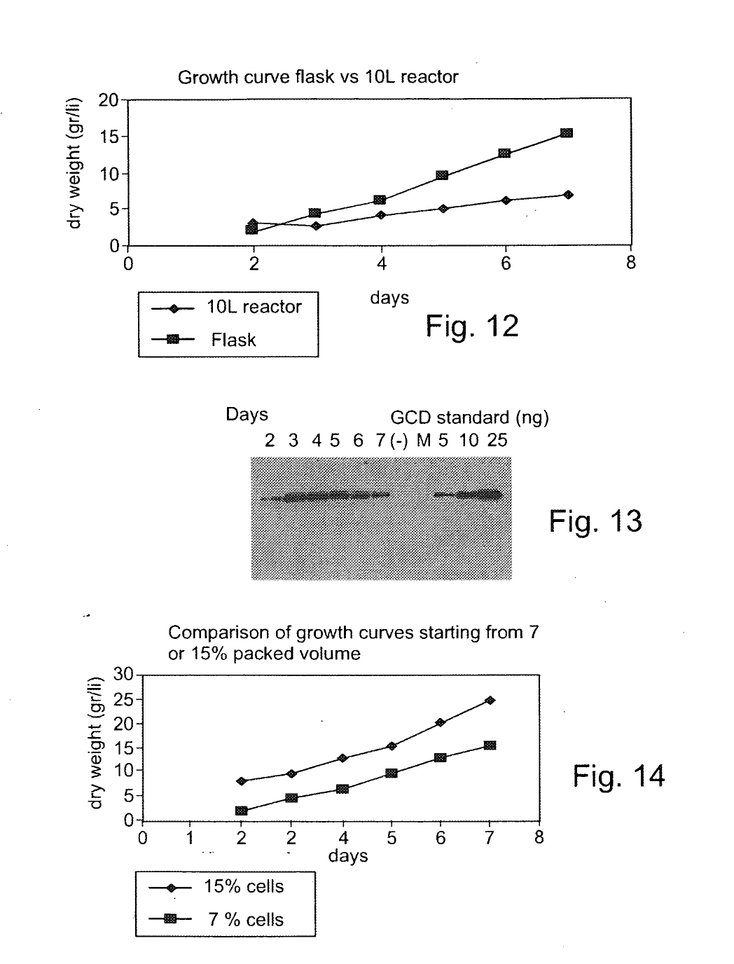

FIG. 12 shows that the cells grown in the flask initially show a higher rate of growth, possibly due to the degree of aeration; however, the rates of growth for cells grown in the device and in the flask were ultimately found to be highly similar, and the experimental results obtained in the below experiments to also be highly similar.

The amount of protein in the transfected plant cells was then measured. GCD was extracted in phosphate buffer 0.5 M pH 7.2 containing 10% w/w PVPP (Poly vinyl poly pyrolidone) and 1% Triton X-100. GCD content was measured in samples from flask grown suspensions and/or with samples taken from cell cultures grown in the device of the present invention, by using quantitative Western blot. The Western blot was performed as follows.

For this assay, proteins from the obtained sample were separated in SDS polyacrylamide gel electrophoresis and transferred to nitrocellulose. For this purpose, SDS polyacrylamide gels were prepared as follows. The SDS gels consist of a stacking gel and a resolving gel (in accordance with Laemmli, UK 1970, Cleavage of structural proteins during assembly of the head of bacteriphage T4, Nature 227, 680-685). The composition of the resolving gels was as follows: 12% acrylamide (Bio-Rad), 4 microliters of TEMED (N,N,N′,N′-tetramethylethylenediamine; Sigma catalog number T9281) per 10 ml of gel solution, 0.1% SDS, 375 mM Tris-HCl, pH 8.8 and ammonium persulfate (APS), 0.1%. TEMED and ammonium persulfate were used in this context as free radical starters for the polymerization. About 20 minutes after the initiation of polymerization, the stacking gel (3% acrylamide, 0.1% SDS, 126 mM Tris-HCl, pH 6.8, 0.1% APS and 5 microliters of TEMED per 5 ml of stacking gel solution) was poured above the resolving gel, and a 12 or 18 space comb was inserted to create the wells for samples.

The anode and cathode chambers were filled with identical buffer solution: Tris glycine buffer containing SDS (Biorad, catalog number 161-0772), pH 8.3. The antigen-containing material was treated with 0.5 volume of sample loading buffer (30 ml glycerol (Sigma catalog number G9012), 9% SDS, 15 ml mercaptoethanol (Sigma catalog number M6250), 187.5 mM Tris-HCl, pH 6.8, 500 microliters bromophenol blue, all volumes per 100 ml sample buffer), and the mixture was then heated at 100° C. for 5 minutes and loaded onto the stacking gel.

The electrophoresis was performed at room temperature for a suitable time period, for example 45-60 minutes using a constant current strength of 50-70 volts followed by 45-60 min at 180-200 Volt for gels of 13 by 9 cm in size. The antigens were then transferred to nitrocellulose (Schleicher and Schuell, Dassel).

Protein transfer was performed substantially as described herein. The gel was located, together with the adjacent nitrocellulose, between Whatmann 3 MM filter paper, conductive, 0.5 cm-thick foamed material and wire electrodes which conduct the current by way of platinum electrodes. The filter paper, the foamed material and the nitrocellulose were soaked thoroughly with transfer buffer (TG buffer from Biorad, catalog number 161-0771, diluted 10 times with methanol and water buffer (20% methanol)). The transfer was performed at 100 volts for 90 minutes at 4° C.

After the transfer, free binding sites on the nitrocellulose were saturated, at 4° C. over-night with blocking buffer containing 1% dry milk (Dairy America), and 0.1% Tween 20 (Sigma Cat P1379) diluted with phosphate buffer (Riedel deHaen, catalog number 30435). The blot strips were incubated with an antibody (dilution, 1:6500 in phosphate buffer containing 1% dry milk and 0.1% Tween 20 as above, pH 7.5) at 37° C. for 1 hour.

After incubation with the antibody, the blot was washed three times for in each case 10 minutes with PBS (phosphate buffered sodium phosphate buffer (Riedel deHaen, catalog number 30435)). The blot strips were then incubated, at room temperature for 1 h, with a suitable secondary antibody (Goat anti rabbit (whole molecule) HRP (Sigma cat # A-4914)), dilution 1:3000 in buffer containing 1% dry milk (Dairy America), and 0.1% Tween 20 (Sigma Cat P1379) diluted with phosphate buffer (Riedel deHaen, catalog number 30435)). After having been washed several times with PBS, the blot strips were stained with ECL developer reagents (Amersham RPN 2209).

After immersing the blots in the ECL reagents the blots were exposed to X-ray film FUJI Super RX 18×24, and developed with FUJI-ANATOMIX developer and fixer (FUJI-X fix cat# FIXRTU 1 out of 2). The bands featuring proteins that were bound by the antibody became visible after this treatment.

FIG. 13 shows the results, indicating that the amount of GCD protein relative to the total protein (plant cell and GCD) was highest on days 3 and 4, after which the relative level of GCD declined again. Results were similar for cells grown in flasks or in the device of the present invention.

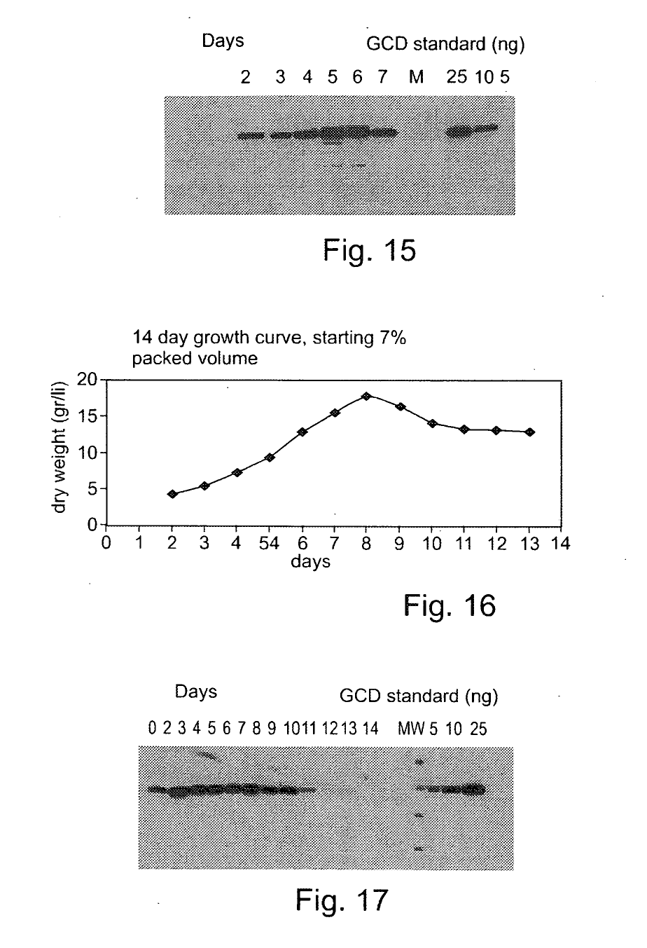

Next, the start point of 7% and 15% packed cell volume were compared (again results were similar for cells grown in flasks or in the device of the present invention). By “packed cell volume” it is meant the volume of cells setttling within the device of the present invention after any disturbing factors have been removed, such as aeration of the media. FIG. 14 shows the growth curves, which are parallel. FIG. 15 shows the amount of GCD protein from a quantitative Western blot, indicating that the amount of GCD protein relative to the total protein (plant cell and GCD) was highest on days 5 and 6, after which the relative level of GCD declined again (it should be noted that samples were taken from cells grown from 15% packed cell volume).

Growth was measured over an extended period of time (14 days) to find the stationary point, where the rate of growth levels off. As shown with regard to FIG. 16, this point is reached on day 8, after which growth is reduced somewhat. Therefore, in order to be able to grow cells transfected with a polynucleotide expressing GCD, preferably cells are grown at least until the stationary point, which in this Example is preferably until day 8 (or shortly thereafter).

FIG. 17 shows that the maximum amount of GCD (relative to other proteins) is produced by transformed cells through day 8, after which the amount of GCD produced starts to decline.

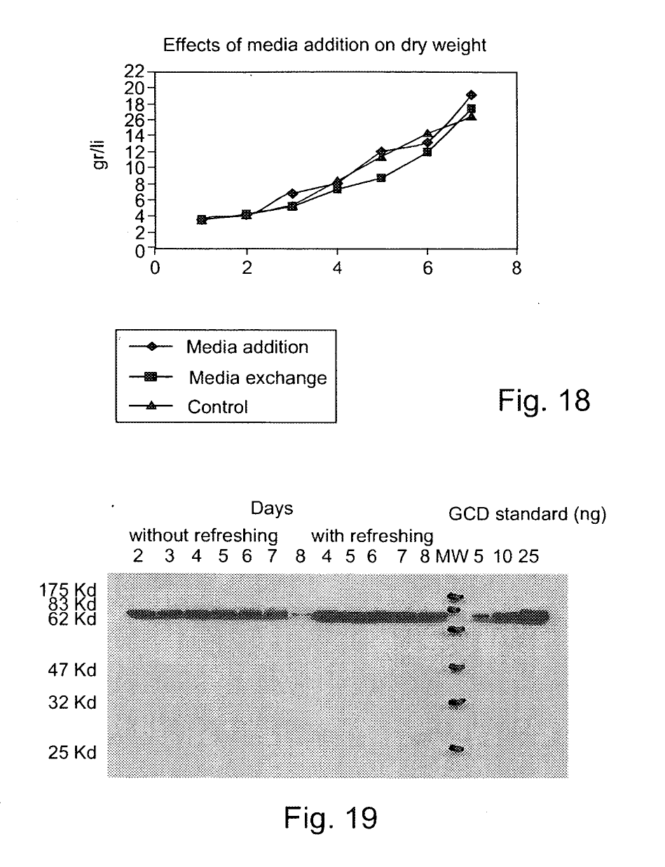

Adding at least some fresh media to the container was found to increase cell growth and the amount of GCD being produced by the cells. As shown with regard to FIG. 18, the addition of fresh (concentrated) media (media addition) and/or replacement of media (media exchange) on the fourth day maintains high growth level of cells beyond day 8. Furthermore, the replacement of media with fresh media on day four clearly enables a much higher amount of GCD to be produced (see FIG. 19 for a quantitative Western blot; “refreshing media” refers to replacement of all media with fresh media). Adding concentrated fresh media on day four also results in a higher amount of GCD being produced (see FIG. 20 for a quantitative Western blot).

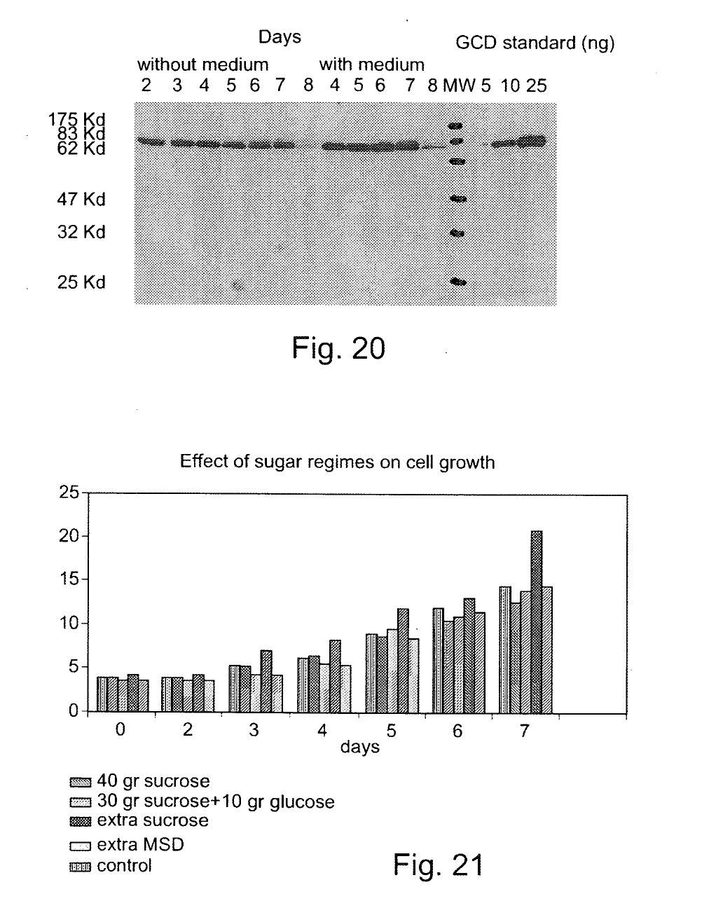

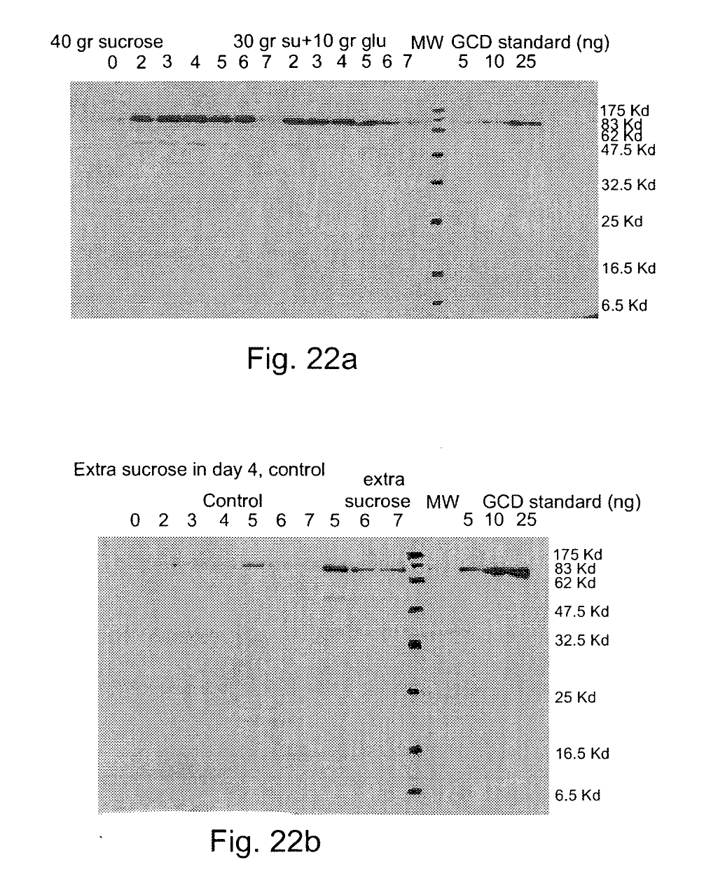

The effect of different sugar regimes on cell growth is shown with regard to FIG. 21, and on production of GCD is shown with regard to FIG. 22. As previously described, optionally but preferably, higher sucrose levels than normally recommended for plant cell culture are used, for example by adding sucrose, such that the concentration in the media may optionally be 40 g/l rather than 30 g/l. One or more other sugars may optionally be added, such as glucose, fructose or other sugars, to complement sucrose. Sucrose (and/or one or more other sugars) is also optionally and preferably added during the cell culture process, more preferably on day 3 or 4 after starting the culture process. The effect of these alterations to the cell culture process is described in greater detail below.

In FIG. 21, the label 40 g sucrose indicates that 40 g of sucrose was added at the start of cell growth; the label “30 g sucrose+10 g glucose” indicates that this combination of sugars was present at the start of cell growth; the label “extra sucrose” indicates that 30 g/l of sucrose was present at day zero (start of cell growth) and that 30 g/l sucrose was added to the medium on day 4; the label “extra MSD” indicates that MSD medium was added; and the label “control” indicates that 30 g/l sucrose was present at day zero (start of cell growth). As shown, the presence of extra MSD had the greatest effect by day 7, followed by the use of a higher amount of sucrose (40 g/l), followed by the addition of sucrose mid-way through the growth cycle.

FIG. 22 shows that both the use of a higher amount of sucrose (40 g/l) in FIG. 22A and the addition of sucrose on day four (FIG. 22B) increased the amount of GCD produced; however, the latter condition produced a spike of GCD production on day 5, while the former condition provided overall higher amounts of GCD production for several days.

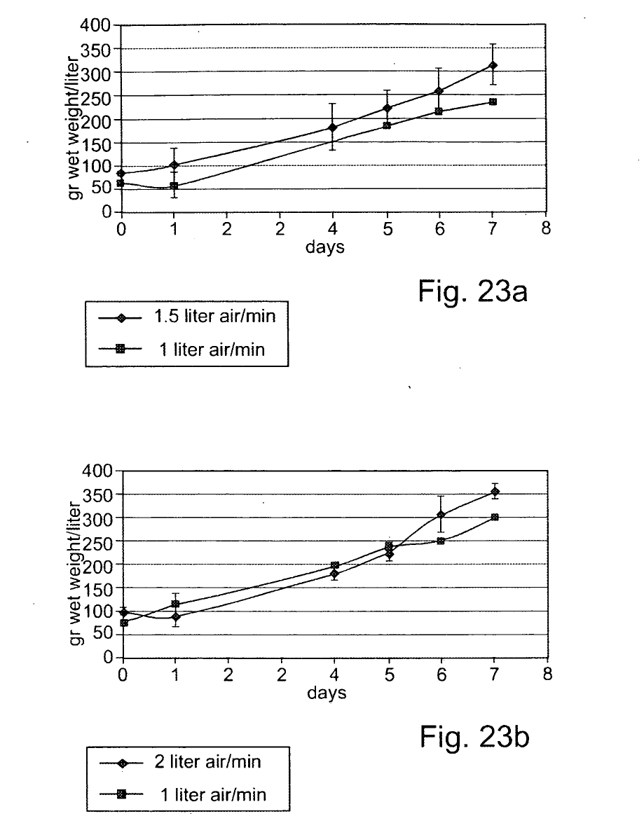

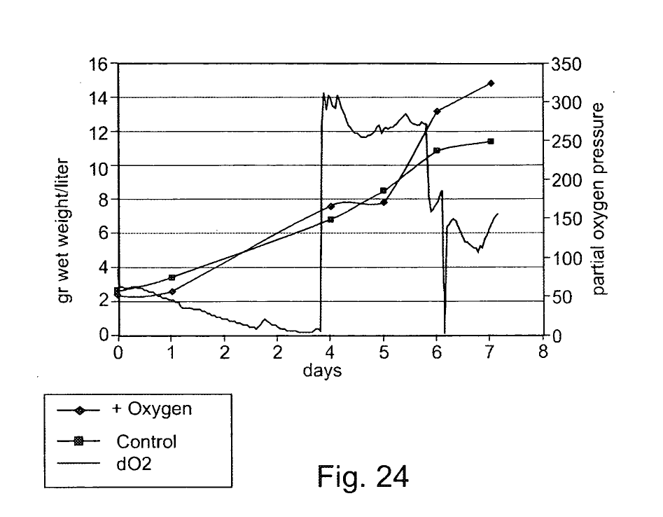

Increased aeration generally (i.e.—the presence of a more rapid gas exchange) and increased oxygen specifically both increased the rate of growth of GCD transformed plant cells. For these experiments, the cultures were initially aerated at a rate of 1 liter of air per minute. Increased aeration was performed by increasing the rate of air flow to 1.5 or 2 liters per minute, as shown with regard to FIG. 23. Oxygen was added starting on the fourth day, with up to 300% oxygen added as shown with regard to FIG. 24 (solid line without symbols shows the oxygen pressure). Otherwise the conditions were identical.

FIG. 23 shows the effect of aeration rate on cell growth in a 10 L device according to the present invention. As shown, increased aeration (greater than the base of 1 L air exchange per minute), provided as 1.5 L per minute (FIG. 23A) or 2 L per minute (FIG. 23B) resulted in an increased level of cell growth.

FIG. 24 shows the effect of adding more oxygen to the device according to the present invention. Oxygen was added starting on day 4; the pressure of the additional oxygen is shown as a solid black line without symbols. It should be noted that because the cell culture medium becomes increasingly viscous as the cells grow and multiply, the measurement of oxygen pressure can be somewhat variable, even though the flow of oxygen was maintained at a constant level. As shown, cells receiving extra oxygen clearly showed a higher growth rate, particularly after day 7, when the growth rate typically starts to level off, as shown for cells which did not receive oxygen.

1. A disposable device for axenically culturing and harvesting cells and/or tissue in at least one cycle, said device comprising a sterilisable disposable container having a top end and a bottom end, which container may be at least partially filled with a suitable sterile biological cell and/or tissue culture medium and/or axenic inoculant and/or sterile air and/or required other sterile additives, said container comprising: (i) a gas outlet for removing excess air and/or waste gases from said container; (ii) an additive inlet for introducing said inoculant and/or said culture medium and/or said additives into said container; and characterized in further comprising (iii) a reusable harvester comprising a flow controller for enabling harvesting of at least a desired portion of said medium containing cells and/or tissues when desired, thereby enabling said device to be used continuously for at least one further consecutive culturing/harvesting cycle, wherein a remainder of said medium containing cells and/or tissue, remaining from a previous harvested cycle, may serve as inoculant for a next culture and harvest cycle, wherein said culture medium and/or said required additives are provided.

2. The device of claim 1, wherein said disposable container is transparent and/or translucent.

3. The device of claim 1, further comprising an air inlet for introducing sterile gas in the form of bubbles into said culture medium through a first inlet opening, wherein said air inlet is connectable to a suitable gas supply.

4. The device of claim 3, wherein said air inlet is for introducing sterile gas more than once during culturing.

5. The device of claim 4, wherein said air inlet is for continuously introducing sterile gas.

6. The device of claim 4, wherein a plurality of different gases are introduced at different times and/or concentrations through said air inlet.

7. The device of claim 1, said harvester comprising a contamination preventer for substantially preventing introduction of contaminants into said container via said harvester.

8. The device of claim 1, wherein said container is non-rigid.

9. The device of claim 8, wherein said container is made from a non-rigid plastic material.

10. The device of claim 9, wherein said material is selected from the group comprising polyethylene, polycarbonate, a copolymer of polyethylene and nylon, PVC and EVA.

11. The device of claim 9, wherein said container is made from a laminate of more than one layer of said materials.

12. The device of claim 9, wherein said container is formed by fusion bonding two suitable sheets of said material along predetermined seams.

13. The device of claim 3, wherein said air inlet comprises an air inlet pipe extending from said inlet opening to a location inside said container at or near said bottom end thereof.

14. The device of claim 3, wherein said at least one air inlet comprises a least one air inlet pipe connectable to a suitable air supply and in communication with a plurality of secondary inlet pipes, each said secondary inlet pipe extending to a location inside said container, via a suitable inlet opening therein, for introducing sterile air in the form of bubbles into said culture medium.

15. The device of claim 14, wherein said device comprises a substantially box-like geometrical configuration, having an overall length, height and width.

16. The device of claim 15, wherein the height-to-length ratio is between about 1 and about 3, and preferably about 1.85.

17. The device of claim 15, wherein the height to width ratio is between about 5 and about 30, and preferably about 13.

18. The device of claim 16, wherein said device comprises a support aperture substantially spanning the depth of said device, said aperture adapted to enable said device to be supported on a suitable pole support.

19. The device of claim 14, further comprising a support structure for supporting said device.

20. The device of claim 19, wherein said support structure comprises a pair of opposed frames, each said frame comprising upper and lower support members spaced by a plurality of substantially parallel vertical support members suitably joined to said upper and lower support members.

21. The device of claim 20, wherein said plurality of vertical support members consists of at least one said vertical support member at each longitudinal extremity of said upper and lower support members.

22. The device of claim 20, wherein said frames are spaced from each other by a plurality of spacing bars releasably or integrally joined to said frames.

23. The device of claim 21, wherein said spacing bars are strategically located such that said device may be inserted and removed relatively easily from said support structure.

24. The device of claim 20, wherein said lower support member of each said frame comprises at least one lower support adapted for receiving and supporting a corresponding portion of said bottom end of said device.

25. The device of claim 24, wherein each said lower support is in the form of suitably shaped tab projecting from each of the lower support members in the direction of the opposed frame.

26. The device of claim 20, wherein said frames each comprise at least one interpartitioner projecting from each frame in the direction of the opposed frame, for to pushing against the sidewall of said device at a predetermined position, such that opposed pairs of said interpartitioner effectively reduce the width of said device at said predetermined position.

27. The device of claim 26, wherein said interpartitioner comprise suitable substantially vertical members spaced from said upper and lower support members in a direction towards the opposed frame with suitable upper and lower struts.

28. The device of claim 19, wherein, said support structure comprises a plurality of castors for transporting said devices.

29. The device of claim 3, wherein at least some of said air bubbles comprise a mean diameter of between about 1 mm and about 10 mm.

30. The device of claim 3, wherein at least some of said air bubbles comprise a mean diameter of about 4 mm.

31. The device of claim 1, wherein said container comprises a suitable filter mounted on said gas outlet for substantially preventing introduction of contaminants into said container via said gas outlet.

32. The device of claim 1, wherein said container further comprises a suitable filter mounted on said additive inlet for substantially preventing introduction of contaminants into said container via said additive inlet.

33. The device of claim 1, further comprising a contamination preventer comprising a U-shaped fluid trap, wherein one arm thereof is aseptically mounted to an external outlet of said harvester by suitable aseptic connector.

34. The device of claim 1, wherein said harvester is located at the bottom of said bottom end of said container.

35. The device of claim 1, wherein said harvester is located near the bottom of said bottom end of said container, such that at the end of each harvesting cycle said remainder of said medium containing cells and/or tissue automatically remains at said bottom end of said container up to a level below the level of said harvester.

36. The device of claim 1, wherein said remainder of said medium containing cells and/or tissue is determined at least partially according to a distance d2 from the bottom of said container to said harvester.

37. The device of claim 1, wherein said remainder of said medium containing cells and/or tissue comprises from about 2.5% to about 45% of the original volume of said culture medium and said inoculant.

38. The device of claim 37, wherein said remainder of said medium containing cells and/or tissue comprises from about 10% to about 20% of the original volume of said culture medium and said inoculant.

39. The device of claim 1, wherein said bottom end is substantially convex.

40. The device of claim 1, wherein said bottom end is substantially frusta-conical.

41. The device of claim 1, wherein said container comprises an internal fillable volume of between about 5 liters and about 200 liters, preferably between about 50 liters and 150 liters, and preferably about 100 liters.

42. The device of claim 1, wherein said device further comprises suitable attacher for attaching said device to a suitable support structure.

43. The device of claim 42, wherein said attacher comprises a loop of suitable material preferably integrally attached to said top end of said container.

44. The device of claim 1, adapted to plant cell culture.

45. The device of claim 44, wherein said plant cell culture comprises plant cells obtained from a plant root.

46. The device of claim 45, wherein said plant root is selected from the group consisting of Agrobacterium rihzogenes transformed root cell, celery cell, ginger cell, horseradish cell and carrot cell.

47. A battery of said devices, comprising at least two said disposable devices of claim 3.

48. The battery of claim 47, wherein said devices are supported by a suitable support structure via an attacher of each said device.

49. The battery of claim 47, wherein said gas outlet of each said device is suitably connected to a common gas outlet piping which optionally comprises a blocker for preventing contaminants from flowing into said devices.

50. The battery of claim 49, wherein said blocker comprises a suitable filter.

51. The battery of claim 47, wherein said additive inlet of each said device is suitably connected to a common additive inlet piping having a free end optionally comprising suitable aseptic connector thereat.

52. The battery of claim 51, wherein said free end is connectable to a suitable supply of medium and/or additives.

53. The battery of claim 47, wherein said harvester of each said device is suitably connected to a common harvesting piping having a free end optionally comprising suitable aseptic connector thereat.

54. The battery of claim 53, further comprising contamination preventer for substantially preventing introduction of contaminants into said container via said common harvesting piping.

55. The battery of claim 54, wherein said contamination preventer comprises a U-shaped fluid trap, wherein one arm thereof is free having an opening and wherein the other end thereof is aseptically mountable to said free end of said common harvesting piping via suitable aseptic connector.

56. The battery of claim 55, wherein said free end of said U-tube is connectable to a suitable receiving tank.

57. The battery of claim 47, wherein said air inlet of each said device is suitably connected to a common air inlet piping having a free end optionally comprising suitable aseptic connector thereat.

58. The battery of claim 57, wherein said free end is connectable to a suitable air supply.

59. A method for axenically culturing and harvesting cells and/or tissue in a disposable device comprising:
providing said device which comprises a sterilisable transparent and/or translucent disposable container having a top end and a bottom end, which container may be at least partially filled with a suitable sterile biological cell and/or tissue culture medium and/or axenic inoculant and/or sterile air and/or other sterile required additives, said container comprising:
(i) gas outlet for removing excess air and/or waste gases from said container;
(ii) additive inlet for introducing said inoculant and/or said culture medium and/or said additives into said container;
(iii) reusable harvester comprising suitable flow controller for enabling harvesting of at least a portion of said medium containing cells and/or tissue when desired, thereby enabling said device to be used continuously for at least one further consecutive cycle, wherein a remainder of said medium containing cells and/or tissue, remaining from a previously harvested cycle may serve as inoculant for a next culture and harvest cycle, wherein said culture medium and/or said required additives are provided;
providing axenic inoculant via said harvester;
providing sterile said culture medium and/or, sterile said additives via said additive inlet;
optionally illuminating said container with external light; and
allowing said cells and/or tissue to grow in said medium to a desired yield.

60. The method of claim 59, further comprising:
allowing excess air and/or waste gases to leave said container continuously via said gas outlet.

61. The method of claim 60, further comprising:
checking for contaminants and/or the quality of the cells/tissues which are produced in said container: if contaminants are found or the cells/tissues which are produced are of poor quality, the device and its contents are disposed of;
if contaminants are not found, harvesting said desired portion of said medium containing cells and/or tissue.

62. The method of claim 61, wherein while harvesting said desired portion, leaving a remainder of medium containing cells and/or tissue in said container, wherein said remainder of medium serves as inoculant for a next culture/harvest cycle.

63. The method of claim 62, further comprising:
providing sterile said culture medium and/or sterile said additives for the next culture/harvest cycle via said additive inlet; and
repeating the growth cycle until said contaminants are found or the cells/tissues which are produced are of poor quality, whereupon the device and its contents are disposed of.

64. The method of claim 59, wherein said device further comprises an air inlet for introducing sterile air in the form of bubbles into said culture medium through a first inlet opening connectable to a suitable sterile air supply, said method further comprising the step of providing sterile air to said air inlet during the first and each subsequent cycle.

65. The method of claim 64, wherein said sterile air is supplied continuously throughout at least one culturing cycle.

66. The method of claim 64, wherein said sterile air is supplied in pulses during at least one culturing cycle.

67. A method for axenically culturing and harvesting cells and/or tissue in a battery of disposable devices comprising:
providing a battery of devices of claim 55, and for at least one said device thereof:
providing axenic inoculant to said device via a common harvesting piping;
providing sterile said culture medium and/or sterile additives to said device via common additive inlet piping;
optionally illuminating said device with external light; and
allowing said cells and/or tissue in said device to grow in said medium to a desired yield.

68. The method of claim 67, further comprising:
allowing excess air and/or waste gases to leave said device continuously via common gas outlet piping;
checking for contaminants and/or the quality of the cells/tissues which are produced in said device: if in said device contaminants are found or the cells/tissues which are produced are of poor quality, said harvester of said device is closed off preventing contamination of other said devices of said battery;
if in all of said devices of said battery contaminants are found or the cells/tissues which are produced therein are of poor quality, all the devices and their contents are disposed of;
if contaminants are not found and the quality of the produced cells/tissues is acceptable, for each harvestable device, harvesting a desired portion of said medium containing cells and/or tissue via common harvesting piping and said contamination preventer to a suitable receiving tank.

69. The method of claim 68, wherein a remainder of medium containing cells and/or tissue remains in said container, wherein said remainder serves as inoculant for a next culture/harvest cycle; and the method further comprises:
providing sterile said culture medium and/or sterile said additives for the next culture/harvest cycle via said additive inlet to form a growth cycle.

70. The method of claim 69, wherein said growth cycle is repeated until said contaminants are found or the cells/tissues which are produced are of poor quality for all of said devices of said battery, whereupon said contamination preventer is disconnected from a common harvester and said devices and their contents are disposed of.

71. A method for axenically culturing and harvesting cells and/or tissue in a battery of disposable devices comprising:
providing a battery of devices of claim 58, and for at least one said device thereof:
providing axenic inoculant to said device via common harvesting piping;
providing sterile said culture medium and/or sterile additives to said device via common additive inlet piping;
providing sterile air to said device via common air inlet piping;
optionally illuminating said device with external light; and
allowing said cells and/or tissue in said device to grow in said medium to a desired yield.

72. The method of claim 71, further comprising:
allowing excess air and/or waste gases to leave said device continuously via common gas outlet piping; and
checking for contaminants and/or the quality of the cells/tissues which are produced in said device: if in said device contaminants are found or the cells/tissues which are produced are of poor quality, said harvester of said device is closed off preventing contamination of other said devices of said battery; if in all of said devices of said battery contaminants are found or the cells/tissues which are produced therein are of poor quality, all the devices and their contents are disposed of, if contaminants are not found and the quality of the produced cells/tissues is acceptable, the device is considered harvestable.

73. The method of claim 72, further comprising:
harvesting at least a desired portion of said medium containing cells and/or tissue for each harvestable device via common harvesting piping and said contamination preventer to a suitable receiving tank.

74. The method of claim 73, wherein a remainder of medium containing cells and/or tissue remains in said container, wherein said remainder serves as inoculant for a next culture/harvest cycle; and the method further comprises:
providing sterile said culture medium and/or sterile said additives for the next culture/harvest cycle via said additive inlet to form a growth cycle.

75. The method of claim 74, wherein said growth cycle is repeated until said contaminants are found or the cells/tissues which are produced are of poor quality for all of said devices of said battery, whereupon said contamination preventer is disconnected from a common harvester and said devices and their contents are disposed of.

76. A device for plant cell culture, comprising a disposable container for culturing plant cells.

77. The device of claim 76, wherein said disposable container is capable of being used continuously for at least one further consecutive culturing/harvesting cycle.

78. The device of claim 77, further comprising:
a reusable harvester comprising a flow controller for enabling harvesting of at least a desired portion of medium containing cells and/or tissues when desired, thereby enabling said device to be used continuously for at least one further consecutive culturing/harvesting cycle.

79. The device of claim 78, wherein said flow controller maintains sterility of a remainder of said medium containing cells and/or tissue, such that said remainder of said medium remaining from a previous harvested cycle, serves as inoculant for a next culture and harvest cycle.

80. A method for culturing plant cells, comprising:
culturing plant cells in a disposable container.

81. The method of claim 80, wherein said disposable container comprises an air inlet for introducing sterile gas or a combination of gases.

82. The method of claim 81, wherein said sterile gas comprises air.

83. The method of claim 82, wherein said sterile gas combination comprises a combination of air and additional oxygen.

84. The method of claim 83, wherein said additional oxygen is added separately from said air.

85. The method of claim 84, wherein said additional oxygen is added a plurality of days after initiating cell culture.

86. The method of claim 81, wherein said sterile gas or combination of gases is added more than once during culturing.

87. The method of claim 81, wherein said air inlet is for continuously introducing sterile gas.

88. The method of claim 81, wherein a plurality of different gases is introduced at different times and/or concentrations through said air inlet.

89. The method of claim 81, further comprising:
aerating said cells through said inlet.

90. The method of claim 89, wherein said aerating comprises administering at least 1.5 L gas per minute.

91. The method of claim 80, further comprising:
providing sufficient medium for growing said cells.

92. The method of claim 91, wherein sufficient medium is at a concentration of at least about 125% of a normal concentration of medium.

93. The method of claim 91, further comprising:
adding media during growth of the cells but before harvesting.

94. The method of claim 93, further comprising:
adding additional media at least about 3 days after starting culturing said cells.

95. The method of claim 93, further comprising:
replacing media completely at least about 3 days after starting culturing said cells.

96. The method of claim 91, wherein said medium comprises a mixture of sugars.

97. The method of claim 91, wherein said medium comprises a larger amount of sucrose than normal for cell culture.

98. The method of claim 80, wherein said plant cells produce a recombinant protein.
